(19) United States
(12) Patent Application Publication
(10) Pub. No.: US 2026/0347615 A1
(43) Pub. Date: Nov. 5, 2026

(54) COLORANT COMPOSITIONS AND METHODS FOR PRODUCTION

(71) Applicant: MICHROMA CORPORATION, Wilmington, DE (US)

(72) Inventors: Mauricio BRAIA, Wilmington, DE (US); Sebastián SUÁREZ MUÑOZ, Wilmington, DE (US); Emilia MAZZA, Wilmington, DE (US); Joaquín BERMEJO, Wilmington, DE (US)

(73) Assignee: MICHROMA CORPORATION, Wilmington, DE (US)

(21) Appl. No.: 19/117,925

(22) PCT Filed: Oct. 2, 2023
(86) PCT No.: PCT/US2023/075757

§ 371 Date: Apr. 2, 2025

Related U.S. Application Data
(60) Provisional application No. 63/378,113, filed on Oct. 3, 2022.

(51) Int. Cl. C09B 61/00; C12N 1/14; C12R 1/80

(52) U.S. Cl. CPC C09B 61/00; C12N 1/14; C12R 2001/80

(57) ABSTRACT

Disclosed are methods of culturing a pigment producing fungus, including steps of inoculating a first liquid media with conidia of the fungus; performing a mycelium production culturing step comprising culturing the conidia in the first liquid media to generate an inoculum culture comprising mycelium; inoculating a second liquid media with the inoculum culture or a portion thereof; and performing a pigment production culturing step comprising culturing the mycelium in the second liquid media to generate a pigment-producing mycelium culture; wherein the pigment-producing fungus is from the genus Talaromyces, Penicillium, or Monascus, and wherein the first liquid media, the second liquid media, or both lack a phosphate source. Also disclosed herein are compositions produced by the culturing methods disclosed herein.

### CROSS-REFERENCE

This application claims the benefit of U.S. Provisional Application No. 63/378,113, filed Oct. 3, 2022, the content of which is incorporated by reference herein in its entirety.

### BACKGROUND

There is a demand for natural colorants for products such as food, medicine, and cosmetics. Suitable colorants need to provide the desired color characteristics while also having appropriate safety, nutritional, and flavor properties. Additionally, colorants should preferably be natural as opposed to synthetic compounds. Microorganisms are currently utilized to produce industrially useful natural colorants for the food industry.

The filamentous fungus Talaromyces atroroseus is known to produce red pigments. Such pigments include compounds known as monascorubraminic acids, which are azaphilone compounds and include red, orange, and yellow pigments. Known production methods of monascorubraminic acids by culturing T. atroroseus have low efficiency and produce low yields of individual pigments which are not suitable for a broad range of uses in products such as food, cosmetics, and medicine. New colorant compositions and methods for their production are needed in order to reduce input resources, improve colorant production output, and provide desirable color, stability, solubility, and other properties for use in a wide range of products.

### SUMMARY

Aspects of the present disclosure provide methods of culturing pigment-producing fungus and production of colorant compositions. In some embodiments, the method comprises culturing a pigment-producing fungus in a liquid media that lacks a phosphate source. In some embodiments, the pigment-producing fungus is from the genus Talaromyces, Penicillium, or Monascus. In some embodiments, the pigment-producing fungus is selected from the group consisting of Talaromyces atroroseus, Talaromyces verruculosus, Talaromyces albobiverticillius, Talaromyces purpureogenus, Talaromyces amestolkiae, Talaromyces ruber, Talaromyces flavus, Penicillium purpurogenum, Penicillium oxalicum, Penicillium armenica, Penicillium marneffei, Penicillium atrovenetum, Penicillium rubrum, Monascus purpureus, Monascus ruber, and Monascus pilosus.

In some embodiments, the liquid media comprises a mixed nitrogen source. In some embodiments, the mixed nitrogen source is peptone, tryptone, soy tryptone, yeast extract, one or more proteins, one or more peptides, or two or more amino acids. In some embodiments, the pH is decreased during the culturing. In some embodiments, the pH is decreased to about 4.5, about 4.0, about 3.5, about 3.0, or about 2.5 during the culturing.

In some embodiments, the method further comprises inoculating the liquid media with mycelium of the pigment-producing fungus.

Also disclosed herein is a method for culturing a pigment-producing fungus, the method comprising the steps of: inoculating a first liquid media with conidia of the fungus; performing a mycelium production culturing step comprising culturing the conidia in the first liquid media to generate an inoculum culture comprising mycelium; inoculating a second liquid media with the inoculum culture or a portion thereof, and performing a pigment production culturing step comprising culturing the mycelium in the second liquid media to generate a pigment-producing mycelium culture; wherein the pigment-producing fungus is from the genus Talaromyces, Penicillium, or Monascus, and wherein the first liquid media, the second liquid media, or both lack a phosphate source. In some embodiments, the fungus is selected from the group consisting of Talaromyces atroroseus, Talaromyces verruculosus, Talaromyces albobiverticillius, Talaromyces purpureogenus, Talaromyces amestolkiae, Talaromyces ruber, Talaromyces flavus, Penicillium purpurogenum, Penicillium oxalicum, Penicillium armenica, Penicillium marneffei, Penicillium atrovenetum, Penicillium rubrum, Monascus purpureus, Monascus ruber, and Monascus pilosus.

In some embodiments, the first liquid media, the second liquid media, or both comprise a mixed nitrogen source. In some embodiments, the first liquid media comprises a first mixed nitrogen source. In some embodiments, the first mixed nitrogen source is selected from the group consisting of peptone, tryptone, soy tryptone, yeast extract, one or more proteins, one or more peptides, and two or more amino acids. In some embodiments, the second liquid media comprises a second mixed nitrogen source. In some embodiments, the second mixed nitrogen source is selected from the group consisting of peptone, tryptone, soy tryptone, yeast extract, one or more proteins, one or more peptides, and two or more amino acids.

In some embodiments, the pH is decreased during the pigment production culturing step. In some embodiments, the pH is decreased to about 4.5, about 4.0, about 3.5, about 3.0, or about 2.5 during the pigment production culturing step.

In some embodiments, the dissolved oxygen is maintained at about 30% to 35% in the mycelium production culturing step. In some embodiments, the dissolved oxygen is maintained at about 30% to 35% in the pigment production culturing step.

In some embodiments, a second nitrogen source is provided during the pigment production culturing step. In some embodiments, the second nitrogen source is one or more amino acids.

In some embodiments, the pigment production culturing step is performed in a fermenter.

In some embodiments, the mycelium production culturing step continues for 24 to 36 hours.

In some embodiments, the method further comprises diluting the inoculum culture before the inoculating of step (c). In some embodiments, the inoculum culture is diluted to 0.4× to 0.6×.

In some embodiments, the second liquid media comprises starch. In some embodiments, the starch is depleted to below 0.1 g/L during the pigment production culturing step. In some embodiments, the second liquid media is stirred at a first stirring speed during the pigment production culturing step. In some embodiments, the method further comprises stirring the second liquid media at a second stirring speed after the starch is depleted to below 0.1 g/L during the pigment production culturing step, wherein the second stirring speed is higher than the first stirring speed. In some embodiments, the second stirring speed is 100 to 300 rpm higher than the first stirring speed.

In some embodiments, at the beginning of the pigment production culturing step the second liquid media comprises a sugar and a starch in a ratio of 1:4 to 1:2. In some embodiments, at the beginning of the pigment production culturing step the second liquid media comprises a sugar at 8 to 12 g/L, a starch at 25 to 35 g/L, and yeast extract at 0.8 to 1.2 g/L.

Also disclosed herein is a method for culturing a pigment-producing fungus, the method comprising the steps of: inoculating a first liquid media with conidia of the fungus; performing a mycelium production culturing step comprising culturing the conidia in the first liquid media to generate an inoculum culture comprising mycelium; inoculating a second liquid media with the inoculum culture or a portion thereof, and performing a pigment production culturing step comprising culturing the mycelium in the second liquid media to generate a mycelium culture; wherein the pigment-producing fungus is from the genus Talaromyces, Penicillium, or Monascus, and wherein the second liquid media comprises from 0 to 40 mg/L of phosphate during the second culturing step. In some embodiments, the fungus is selected from the group consisting of Talaromyces atroroseus, Talaromyces verruculosus, Talaromyces albobiverticillius, Talaromyces purpureogenus, Talaromyces amestolkiae, Talaromyces ruber, Talaromyces flavus, Penicillium purpurogenum, Penicillium oxalicum, Penicillium armenica, Penicillium marneffei, Penicillium atrovenetum, Penicillium rubrum, Monascus purpureus, Monascus ruber, and Monascus pilosus.

In some embodiments, the second liquid media comprises phosphate. In some embodiments, the concentration of phosphate is reduced to below 1 mg/L during the pigment production culturing step. In some embodiments, the phosphate is completely depleted from the second liquid media during the pigment production culturing step.

In some embodiments, the pigment production culturing step is continued for at least 24 hours after the phosphate is completely depleted from the second liquid media. In some embodiments, the first liquid media, the second liquid media, or both comprise a mixed nitrogen source. In some embodiments, the first liquid media comprises a first mixed nitrogen source. In some embodiments, the first mixed nitrogen source is selected from the group consisting of peptone, tryptone, soy tryptone, yeast extract, one or more proteins, one or more peptides, and two or more amino acids. In some embodiments, the second liquid media comprises a second mixed nitrogen source. In some embodiments, the second mixed nitrogen source is selected from the group consisting of peptone, tryptone, soy tryptone, yeast extract, one or more proteins, one or more peptides, and two or more amino acids.

In some embodiments, pH is decreased during the pigment production culturing step. In some embodiments, the pH is decreased to about 4.5, about 4.0, about 3.5, about 3.0, or about 2.5 during the pigment production culturing step.

In some embodiments, the dissolved oxygen is maintained at about 30% to 35% in the mycelium production culturing step. In some embodiments, the dissolved oxygen is maintained at about 30% to 35% in the pigment production culturing step.

In some embodiments, a second nitrogen source is provided during the pigment production culturing step. In some embodiments, the second nitrogen source is one or more amino acids. In some embodiments, the pigment production culturing step is performed in a fermenter.

In some embodiments, the mycelium production culturing step continues for 24 to 36 hours.

In some embodiments, the method further comprises diluting the inoculum culture before the inoculating of step (c). In some embodiments, the inoculum culture is diluted to 0.4× to 0.6×.

In some embodiments, the second liquid media comprises starch. In some embodiments, the starch is depleted to below 0.1 g/L during the pigment production culturing step. In some embodiments, the second liquid media is stirred at a first stirring speed during the pigment production culturing step. In some embodiments, the method further comprises stirring the second liquid media at a second stirring speed after the starch is depleted to below 0.1 g/L during the pigment production culturing step, wherein the second stirring speed is higher than the first stirring speed. In some embodiments, the second stirring speed is 100 to 300 rpm higher than the first stirring speed.

In some embodiments, at the beginning of the pigment production culturing step the second liquid media comprises a sugar and a starch in a ratio of 1:4 to 1:2. In some embodiments, at the beginning of the pigment production culturing step the second liquid media comprises a sugar at 8 to 12 g/L, a starch at 25 to 35 g/L, and yeast extract at 0.8 to 1.2 g/L.

In some embodiments, the method further comprises the step of extracting one or more pigments from the mycelium culture to obtain a colorant extract. In some embodiments, the one or more pigments are selected from the group consisting of N-glutaryl monascorubraminic acid, N-asparagyl monascorubraminic acid, N-aspartyl monascorubraminic acid, N-cysteinyl monascorubraminic acid, N-phenylalanyl monascorubraminic acid, N-lysyl monascorubraminic acid, N-methionyl monascorubraminic acid, N-glutamyl monascorubraminic acid, and N-arginyl monascorubraminic acid. In some embodiments, the colorant extract is red or a reddish color. Also disclosed is a colorant extract made by a method described above. In some embodiments, the extract is a liquid. In some embodiments, the extract is dried or lyophilized. Also disclosed is a foodstuff comprising the colorant extract. Also disclosed is a cosmetic comprising the colorant extract.

Also provided herein are colorant compositions comprising cis-N-glutaryl monascorubraminic acid and trans-N-glutaryl monascorubraminic acid.

Also described herein is a colorant composition comprising: (a) about 60% to about 75% cis-N-glutaryl monascorubraminic acid; and (b) about 1% to about 7% trans-N-glutaryl monascorubraminic acid.

In some embodiments, the cis-N-glutaryl monascorubraminic acid is present in the colorant composition in an amount of about 65%. In some embodiments, the trans-N-glutaryl monascorubraminic acid is present in the colorant composition in an amount of about 3%.

In some embodiments, the colorant composition further comprises N-glutamyl monascorubraminic acid. In some embodiments, the colorant composition comprises N-glutamyl monascorubraminic acid in an amount of about 2% to about 6%. In some embodiments, the N-glutamyl monascorubraminic acid is present in an amount of about 3%.

In some embodiments, the colorant composition further comprises N-glutaryl monascorubramine. In some embodiments, the colorant composition comprises N-glutaryl monascorubramine in an amount of about 1% to about 4%. In some embodiments, the colorant composition comprises N-glutaryl monascorubramine in an amount of about 2%.

In some embodiments, the colorant composition further comprises ash. In some embodiments, the colorant composition comprises ash in an amount of about 3%.

In some embodiments, the colorant composition further comprises succinate. In some embodiments, the colorant composition comprises succinate in an amount of about 2% to about 12%. In some embodiments, the colorant composition comprises succinate in an amount of about 7%.

In some embodiments, the colorant composition comprises a ω angle measured in the CIELAB color space in the range of about 30° to 40°. In some embodiments, the ω angle is about 34°.

In some embodiments, the colorant composition comprises a L* value of about 75 to about 85. In some embodiments, the L* value is about 78.

In some embodiments, the colorant composition comprises an a* value of about 15 to about 25. In some embodiments, the a* value is about 22.

In some embodiments, the colorant composition comprises a b* value of about 20 to about 30. In some embodiments, the b* value is about 25.

In some embodiments, the colorant composition comprises a C value of about 45 to about 55. In some embodiments, the C value is about 49.

In some embodiments, the coloring capacity of the colorant composition is at least 220.

In some embodiments, the coloring capacity of the colorant composition is about 250.

In some embodiments, provided herein is a colorant composition comprising:
(a) about 65% cis-N-glutaryl monascorubraminic acid;
(b) about 3% trans-N-glutaryl monascorubraminic acid;
(c) about 3% N-glutaryl monascorubraminic acid;
(d) about 3% N-glutamyl monascorubraminic acid; and
(e) about 7% succinate.

In some embodiments, provided herein is a foodstuff comprising the colorant compositions provided herein. In some embodiments, provided herein is a cosmetic comprising the colorant compositions provided herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1: Assessment of the biomass growth and color production in AMYG culture medium over a range of carbon source concentrations. The productivity (Qp), specific productivity (qp), and biomass titer (X) were calculated at each concentration of starch.

FIG. 2: Effect of the inoculum composition on the color production. The productivity (Qp) and specific productivity (qp) were calculated for systems containing AMYG culture medium inoculated with mycelium and conidia.

FIG. 3: Assessment of the biomass growth over different inoculum concentrations in systems containing AMYG culture medium. The biomass titer (X) was calculated at each concentration of inoculum.

FIG. 4: Effect of inoculum size and age on color production in systems containing AYP culture medium. The productivity (Qp) was calculated at each dilution of the inoculum for each incubation time.

FIGS. 5A-5B: Assessment of biomass growth and color production in AMYG culture medium over a range of glutamate concentrations. The productivity (Qp) and specific productivity (qp) were calculated at concentration of glutamate (FIG. 5A). The wavelength of the maximum of absorbance was determined at each glutamate concentration (FIG. 5B).

FIGS. 6A-6B: Assessment of color production in AMYG culture medium over a range of yeast extract concentrations. The productivity (Qp) (FIG. 6A) and specific productivity (qp) (FIG. 6B) were calculated at each concentration of yeast extract (YE).

FIGS. 7A-7B: Assessment of color production in AMYG culture medium over a range of dipotassium phosphate concentrations. The productivity (Qp) (FIG. 7A) and biomass yield (Yxs) (FIG. 7B) were calculated at each concentration of dipotassium phosphate.

FIG. 8: Effect of yeast extract on the color production in systems containing culture medium with and without dipotassium phosphate. The productivity (Qp) and specific productivity (qp) were calculated in the absence (AYP and AMY-PO4 culture media) and presence of dipotassium phosphate (AMYG culture medium).

FIG. 9: Effect of pH on the color production in systems containing AYP culture medium. The productivity (Qp) and biomass titer (X) were calculated at each pH.

FIG. 10: Effect of growth type on the color production in systems containing AYP culture medium (control) and diauxic culture medium. The productivity (Qp) were calculated for each trial and summarized by system. A two-sample Wilcoxon test was used to establish statistical significance in the difference among productive systems (p-value<0.001).

FIGS. 11A-11C: Chemical structures of monascorubraminic acid pigments that may be produced in methods described herein.

FIGS. 12A-D: HPLC chromatogram (FIG. 12A), UV-Vis absorption spectrum (FIG. 12B), MS spectrum (FIG. 12C), and selected reaction monitoring (FIG. 12D) for N-glutaryl monascorubramine.

FIGS. 13A-G: HPLC chromatogram (FIG. 13A), UV-Vis absorption spectrum (FIG. 13B), MS spectrum (FIG. 13C), selected reaction monitoring (FIG. 13D), ¹H NMR spectrum (FIG. 13E), 2D COSY NMR spectrum (FIG. 13F), and 2D HSQC NMR spectrum (FIG. 13G) for cis-N-glutaryl monascorubraminic acid.

FIGS. 14A-F: HPLC chromatogram (FIG. 14A), UV-Vis absorption spectrum (FIG. 14B), MS spectrum (FIG. 14C), ¹H NMR spectrum (FIG. 14D), zoomed ¹H NMR spectrum (FIG. 14F) for trans-N-glutaryl monascorubraminic acid. FIG. 14E shows an overlay of a ¹H NMR spectrum for trans-N-glutaryl monascorubraminic acid and cis-N-glutaryl monascorubraminic acid.

FIGS. 15A-C: HPLC chromatogram (FIG. 15A), UV-Vis absorption spectrum (FIG. 15B), and MS spectrum (FIG. 15C) for N-glutamyl monascorubraminic acid.

FIG. 16: HPLC chromatogram of solvent solution.

FIG. 17: HPLC chromatogram of system suitability mix.

FIG. 18: HPLC chromatogram of colorant composition provided herein detected at 520 nm.

FIG. 19: Base peak chromatogram and absorption at 520 nm for a colorant composition herein.

FIG. 20A-B: HPLC chromatogram of malate, succinate, lactate, formate, acetate, glycerol, and ethanol (FIG. 20A) and HPLC chromatogram of sucrose, glucose, and fructose (FIG. 20B).

### DETAILED DESCRIPTION

While various embodiments of the invention have been shown and described herein, it will be apparent to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” when referring to a number or a numerical range generally means that the number or numerical range referred to is an approximation within experimental variability (or within statistical experimental error), and thus the number or numerical range may vary from, for example, between 1% and 15% of the stated number or numerical range.

Embodiments described herein may comprise or consist of one or more of the elements describes as part of the embodiment. Embodiments within the scope of the present invention may also specifically exclude any element or combination of elements described as part of an embodiment herein.

### I. Culturing Methods

The present disclosure provides methods for culturing pigment-producing fungi, which can be used for producing a colorant composition. The colorant composition may include pigments such as the azaphilone pigments known as monascorubraminic acids. In some embodiments of the culture methods disclosed herein, pigment-producing fungi are cultured in growth media that lacks phosphate or that includes phosphate at very low concentrations. The inventors found that these culture conditions surprisingly produce a high titer of colorant. Due in part to the high titer of colorant, methods disclosed herein may be used for commercial-scale production of colorant suitable for use in food, cosmetics, and other products.

### A. Culture Phases

Culture methods disclosed herein may begin with a phase of producing a seed culture suitable for inoculating a production culture from which a colorant composition may be prepared. The seed culture phase may include inoculation of a liquid media with conidia of the pigment-producing fungus, followed by culturing to produce mycelium of the fungus. The mycelium produced from the seed culture phase may then be used to inoculate liquid media for a production culture. Production of sufficient mycelia suitable for inoculation of a production culture may include multiple passages. For example, liquid media may be inoculated with conidia and cultured until the culture reaches a certain density of mycelium. That mycelium culture may then be used to inoculate additional fresh liquid media in order to increase the volume of mycelium culture available for inoculating a production culture. The culturing steps that take place before inoculation of a production batch may be performed under conditions that optimize for mycelium proliferation rather than pigment production. Conversely, production cultures can be performed under conditions that optimize for production of pigment as opposed to maximizing mycelium proliferation.

The amount of conidia used to inoculate the liquid media in a seed culture process may be 10³, 10⁴, 10⁵, 10⁶, 10⁷, or 10⁸ conidia per ml of the liquid media, or between any two of these values. The seed production process may continue for sufficient time to produce a mycelium culture suitable for inoculating the liquid media in the second step. The first step may continue for 6, 12, 18, 24, 30, 36, 42, 48, 54, 60, 66, or 72 hours, or a range between any two of these values (e.g., 24 to 36, 24 to 30, or 18 to 36 hours). In some embodiments, the seed production culture may continue for 24 to 48 hours. The culturing may continue until the culture reaches stationary phase, until a carbon source, nitrogen source, or phosphate source is depleted from the liquid media, or until productivity stops increasing.

The mycelium culture produced by a seed production process may be used to inoculate the liquid medium in the production culture process. The volume of the mycelium culture inoculum may be 0.1, 0.5, 1, 2, 3, 4, 5, 6, 7, 8, 9, 10, 11, 12, 13, 14, 15, 16, 17, 18, 19, or 20% of the total volume of the liquid medium for the production culture, or a range between any two of these values. The production culturing process may continue for sufficient time for the mycelium to proliferate and produce pigments. A production culture may continue for 6, 12, 18, 24, 30, 36, 42, 48, 54, 60, 66, 72, 84, 96, 108, 120, 132, 144, or 156 hours, or a range between any two of these values. In some embodiments, a production culture step may continue for 72 to 120 hours. In some embodiments, the culturing can continue until production ceases to increase, until a predetermined amount of pigment is produced, or at another pre-determined endpoint.

In some embodiments, a culture comprising mycelium may be diluted before inoculating liquid media for a subsequent culturing step, such as a pigment production culturing step. In some embodiments, the inoculum culture comprising mycelium is diluted to 0.5×. As used herein, a dilution to 0.5× means decreasing the strength of the original culture by half. Diluting a culture to 0.5× thus involves including 1 part of the culture in 2 parts total volume. For example, 1 part water can be added to 1 part culture to make a dilution of 0.5×. Similarly, a dilution to 0.1×

In some embodiments, more than one culture step is included in a process of culturing a pigment-producing fungus. A process can include, for example, 2, 3, 4, 5, 6, 7, 8, 9, 10, or more passaging steps in which a culture of fungus is used to inoculate a culture in a next culturing step. In such embodiments, the conditions of the final culturing step—i.e., a production culture—may be optimized for production of pigments such as by having a low amount or no source of phosphate in the liquid media used in the final culturing step.

In some embodiments, one or more steps of the culturing of the pigment-producing fungus can be performed in a bioreactor or fermenter. For example, a fermenter may be used which includes a vessel in which culture conditions may be monitored and adjusted. Such culture conditions may include oxygen content; agitation; pH; concentrations of nutrient sources such as, for example, sugars, carbohydrates, nitrogen, phosphate; concentrations of metabolites and waste products; concentrations of pigments; and other components. A fermenter may be configured to allow for exchange or addition of liquid media or components thereof during a culturing step. In some embodiments, one or more, or all, of the culturing steps are performed in a fermenter. In some embodiments, one or more, or all, of the culturing steps are performed in a continuous flow manner. In some embodiments, one or more, or all, of the culturing steps are performed in a batch mode, which may be performed in a fermenter or in a culture flask or vessel.

In some embodiments, a pigment-producing fungus is cultured in a single culturing step. The inoculant for the culture may be conidia of the fungus or mycelium of the fungus. The single culturing step may be, for example, a batch culture or a continuous flow culture. The inoculant may be, for example, frozen, lyophilized, or active. For example, the inoculant for the single-step culture process may be conidia or an active culture of mycelium. The liquid media for the single culture step may have a low or limiting amount of phosphate or may be free of a source of phosphate. In some embodiments, the phosphate may be depleted or removed during the culturing step, as described in other embodiments herein.

In some embodiments, the amount of time a culturing step continues is determined by the amount of biomass and/or pigment produced in the culture. For example, in some embodiments, the amount of biomass and/or pigment in the culture may be monitored, and the culture step may be concluded when a desired amount of the biomass and/or pigment is present in the culture. The biomass may then be harvested or the pigment may be extracted to produce a colorant composition.

### B. Culture Media

Culturing methods disclosed herein can use a variety of liquid media suitable for culturing of pigment-producing fungi. Culture media for methods disclosed herein include, among other ingredients, one or more carbon sources or nitrogen sources. In some embodiments, the culture media used for at least part of the culture process lacks a source of phosphate for at least a portion of the culturing time.

In embodiments in which a pigment-producing fungus is cultured in more than one phases (for example, a seed culture phase and production culture phase as discussed above), the liquid media in different culturing phases may be the same or different. In some embodiments, the carbon source or the nitrogen source may be different, or the concentrations of these and other components of the liquid media may be different. In some embodiments, the phosphate concentration is lower in the liquid media used in a production culture step than in a seed culture step or another culturing phase before the production phase. In some embodiments, the liquid media in a seed production phase or another preliminary culturing step includes a source of phosphate, while the liquid media used in a pigment production culturing step does not include a source of phosphate or includes an amount of phosphate that will be entirely or substantially entirely depleted such that no free phosphate ion is detectable in the liquid media or no more than trace amounts of free phosphate ion are detectable in the liquid media. By “free phosphate ion,” it is meant that the phosphate is dissolved in the liquid media and is not incorporated into a cell or macromolecule.

In some embodiments, the liquid media includes a source of phosphate at the beginning of a culturing step, e.g., a pigment production step in which a mycelium culture is used as an inoculant but is depleted of phosphate after culturing for a time. For example, at the beginning of a production culture step, the concentration of phosphate in the liquid media may be at most 1, 5, 10, 20, 30, 40, or 50 mg/ml, or a range between any two of these values, and may be depleted during culturing to 0.0 mg/ml or at most 0.01, 0.05, 0.1, 0.2, 0.3, 0.4, or 0.5 mg/ml, or a range between any two of these values. Such depletion may occur over the course of 1, 2, 3, 4, 5, 6, 7, 8, 9, 10, 12, 14, 16, 18, 20, 22, 24, 26, 28, 30, 32, 34, 36, 38, 40, 42, 44, 46, or 48, 60, 72, 84, 96, 108, 120, 132, 144, or 156 hours or more, or any range between these values. Depletion of the phosphate source may be due to consumption of the phosphate source by the fungus or may be caused by an operator of the culturing process intervening to change the concentration of phosphate in the liquid media. For example, in embodiments in which a culturing step is performed in a fermenter, an operator may exchange or dilute the liquid media such that the amount of phosphate or other nutrient available to the cultured fungus is reduced to a low level, including to zero.

In some embodiments in which phosphate is included in a culture, the phosphate is provided as part of a mixed nutrient source. For example, a mixed nitrogen source such as, for example, peptone, tryptone, or yeast extract, may include some amount of phosphate available for use by the pigment-producing fungus being cultured. In some embodiments, the amount of phosphate provided in a mixed nutrient source, such as, for example, peptone, tryptone, or yeast extract, may be at most 1, 5, 10, 20, 30, 40, or 50 mg/ml, or a range between any two of these values.

A source of phosphate provided in a liquid media may be, for example, inorganic phosphate salts such as potassium phosphates (e.g., monopotassium phosphate), calcium phosphates, sodium phosphates, ammonium phosphates, and magnesium phosphates. In some embodiments, liquid culture media lacks these or any other source of inorganic phosphate. In some embodiments, the culture methods do not include a step of adding an inorganic phosphate source to a liquid media or using a liquid media that includes an inorganic phosphate source. In some embodiments, an inorganic phosphate source is included in one or more liquid culture media. In some embodiments, the initial concentration of the inorganic phosphate source in the liquid culture media is 1, 5, 10, 20, 30, 40, 50, 100, 150, 200, 250, 300, 350, 400, 450, 500, 600, 700, 800, 900, 1000, 1100, 1200, 1300, 1400, or 1500 mg/ml, or a range between any two of these values.

Liquid media used in the methods described herein may comprise a nitrogen source. As used herein, a “mixed nitrogen source” means a source of nitrogen that may be used to support growth of the fungus being cultured that includes more than one distinct nitrogen-containing moiety. For example, one mixed nitrogen source that may be used in methods described herein is peptone, which is a water-soluble mixture of polypeptides and amino acids formed by partial hydrolysis of protein. As another example, a peptide that includes more than one amino acids covalently bonded together provides a mixed nitrogen source in that nitrogen from more than one distinct chemical moiety is available for use by a cultured fungus. In some embodiments of the culture methods, the mixed nitrogen source is peptone, tryptone, soy tryptone, yeast extract, one or more peptides, mixtures of amino acids, or any combination of these. A mixed nitrogen source may also include, for example, any combination of urea, ammonium salt, nitrate salt, and single amino acids such as, for example, glutamate. In some embodiments, the culture steps included in the methods described herein do not use a liquid media that includes only a single nitrogen source, such as a single amino acid. In some embodiments, a single nitrogen source may be added to a liquid media that already includes a mixed nitrogen source, such that the resulting liquid media comprises a mixed nitrogen source. An excess of a single nitrogen source such as, for example, a single amino acid (e.g., glutamate), may be provided in order to encourage production of a particular pigment that incorporates such single amino acid or a metabolite thereof.

Liquid media used in different culturing steps can have different concentrations and mixtures of nitrogen sources. Liquid media used in one culturing step may have more or less of total nitrogen sources than liquid media used in a previous or subsequent culturing step. Liquid media used in one culturing step may include only a single nitrogen source, while liquid media used in a previous or subsequent culturing step may include a mixed nitrogen source or a different single nitrogen source. Nitrogen source concentrations and compositions may also be changed during a culturing step by adding a mixed or single nitrogen source. Such changes can be made in both batch and continuous cultures. A continuous culture process may involve adjusting the concentration or composition of one or more nitrogen sources in the liquid media during a culturing step.

In some embodiments, liquid media used in culturing methods include a total of 0.1, 0.2, 0.3, 0.4, 0.5, 0.6, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 g/L of one or more nitrogen sources, or a range between any two of these values. In some embodiments, liquid culture media includes 0.1, 0.2, 0.3, 0.4, 0.5, 0.6, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or 15.0 g/L of a single nitrogen source such as, for example, urea, ammonium salt, nitrate salt, or a single amino acid, or a range between any two of these values (e.g., 4.0 to 10.0, 5.0 to 9.0, 6.0 to 8.5, 7.0 to 8.5 g/L). In some embodiments, liquid culture media includes 0.1, 0.2, 0.3, 0.4, 0.5, 0.6, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 g/L of a mixed nitrogen source such as, for example, peptone, tryptone, soy tryptone, yeast extract, or one or more peptides, or a range between any two of these values (e.g., 0.1 to 4.0, 0.1 to 3.0, 0.1 to 2.0, 0.1 to 1.0, 0.1 to 0.8, 0.2 to 0.8, 0.2 to 0.7, 0.3 to 0.6). The nitrogen source may have any of the above concentrations at the beginning of a culture step or during a culturing step, and the concentration may be adjusted at the beginning of or during a culturing step.

The pH of liquid media used in some embodiments may be 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, or a range between any two of these values (e.g., 4.0 to 8.0, 5.0 to 7.5, 5.5 to 7.5, 6.0 to 7.0, etc.). In some embodiments, the liquid media may include a buffer to maintain the pH at a desired value or within a desired range. In some embodiments, the liquid media does not include a buffer.

In some embodiments, the pH of the liquid media is maintained throughout the culturing process. In some embodiments, the pH of the liquid media is maintained, such as at pH 5. In some embodiments, the pH of the liquid media is allowed to decrease throughout the culturing followed by an increase in pH by the addition of a base, such as a base as described elsewhere herein (e.g., NaOH). In some embodiments, the pH is increased back to a pH of 5 with the addition of NaOH during culturing. In some embodiments, allowing the liquid media to vary in pH during culturing increases the titer as described in Example 15.

In some embodiments, the dissolved oxygen content of the liquid media during a culturing step is at least 30, 31, 32, 33, 34, or 35%, or is between any two of these values. In some embodiments, the dissolved oxygen of the liquid media during a culturing step is about (e.g., or is decreased to) about 40%. The dissolved oxygen content may be monitored and adjusted in some embodiments including, for example, those that include culture steps in a fermenter. Dissolved oxygen content can also be influenced by the degree of agitation during culturing. In embodiments in which a culture is agitated by rotation, the culture may be rotated at a speed of at least 50, 100, 150, 200, 250, 300, 350, 400, 450, or 500 rpm, or at a speed in a range between any two of these values.

Liquid media used in embodiments disclosed herein may include a suitable carbon source such as, for example, one or more sugars or polysaccharides. The carbon source may be, for example, glucose, sucrose, maltose, soluble starch, beet or cane molasses, malt, or any combination thereof. The carbon source may be present in the liquid media at a concentration of 5, 10, 15, 20, 25, 30, 35, 40, 45, 50, 55, or 60 g/L, or at a concentration range between any two of these values. The concentration ranges for the carbon source as described above may be the concentration in the liquid media at the beginning of a culturing step or during a culturing step. In some embodiments, the concentration of the carbon source may be adjusted during a culturing step or may be maintained at a desired level throughout a culturing step. In some embodiments, the liquid media includes two or more different carbon sources. In some embodiments, the liquid media includes a sugar and a starch. In some embodiments, the sugar and the starch are present in a ratio of about 1:3. In some embodiments, the sugar and the starch are present in a ratio of about 1:5, 1:4, 1:3, 1:2, or 1:1, or a range between any two of these values (e.g., 1:4 to 1:2). In some embodiments, sugar (e.g., glucose) is present at a concentration of 7, 8, 9, 10, 11, or 12 g/L, or a range between any two of these values, and starch is present at a concentration of 25, 26, 27, 28, 29, 30, 31, 32, 33, 34, or 35 g/L, or a range between any two of these values. In some embodiments, a carbon source may be depleted during a culturing step such as, for example, during a pigment production culturing step. In some embodiments, a starch may be depleted during a pigment production culturing step. In some embodiments, a starch may be depleted to below 5, 4, 3, 2, 1, 0.5, 0.1, or 0.01 g/L. In some embodiments, the starch may be depleted from a concentration of 20, 25, 30, or 35 g/L to a concentration below 5, 4, 3, 2, 1, 0.5, 0.1, or 0.01 g/L.

The liquid medium may contain one or more salts. The one or more salts may be trace metal salts. The one or more salts may be selected from magnesium sulphate heptahydrate, dipotassium phosphate, potassium chloride, Iron (II) sulphate heptahydrate, zinc sulphate heptahydrate, manganese chloride tetrahydrate, cobalt chloride hexahydrate, copper sulphate pentahydrate, or ammonium molybdate tetrahydrate. The one or more salts may be selected from KH₂PO₄, NaCl, MgSO₄·7H₂O, KCl, CaCl₂·H₂O and the trace metals may be selected from CuSO₄·5H₂O, Na₂B₄O₇-10H₂O, FeSO₄-7H₂O, MnSO₄—H₂O, Na₂MoO₄-2H₂O, and ZnSO₄-7H₂O. The liquid growth medium may also include one or more compounds selected from ethylenediaminetetraacetic acid (EDTA) or boric acid.

Media may be stirred during culturing steps. The stirring may be, for example, 100, 150, 200, 250, 300, 350, 400, 450, 500, 550, 600, 650, 700, 750, 800, 850, or 900 rpm, or a range between any two of these values. In some embodiments, the stirring speed is maintained constant throughout a culturing step. In some embodiments, the stirring speed is varied during a culturing step. In some embodiments, the stirring speed is changed depending on depletion of a component in a liquid media. In some embodiments, the stirring speed is increased during a pigment production culturing step after a sugar, a starch, a nitrogen source, or a phosphate source is depleted below a certain amount such as, for example, 1, 0.5, 0.1, or 0.01 g/L. In some embodiments, the stirring speed is increased by 50, 100, 150, 200, 250, or 300 rpm, or a range between any two of these values. In some embodiments, the stirring speed is increased by 50, 100, 150, 200, 250, or 300 rpm after a starch in the liquid medium is depleted to below 1, 0.5, 0.1, or 0.01 g/L during a pigment production culturing step.

Liquid media used in embodiments described herein, for example as a liquid media for a pigment production step, may have the following components in the indicated concentrations: glucose at 8 to 12 g/L, starch at 28 to 32 g/L, yeast extract at 0.8 to 1.2 g/L, Fe₂SO₄ at 0.3 to 0.5 g/L, KCl at 0.4 to 0.6 g/L, and MgSO₄ at 4.0 to 6.0 g/L. In some embodiments, the indicated concentrations are the concentrations at the beginning of a culturing step, and the components are depleted over time. In some embodiments, components are maintained at the indicated concentrations (or within 1, 5, 10, or 15% of the indicated concentrations) during a culturing step by addition of components over time.

The culturing may generate about 1.0 to 7.0 grams (g) of the pigments, including monascorubraminic acid pigments, per liter of culture. The culturing may generate at least about 0.5 g, 1.0 g, 1.5 g, 2.0 g, 2.5 g, 3.0 g, 3.5 g, 4.0 g, 4.5 g, 5.0 g, 5.5 g, 6.0 g, 6.5 g, or 7.0 g of pigments per liter of culture, or a range between any two of these values. The culturing may generate at most about 7.0 g, 6.5 g, 6.0 g, 5.5 g, 5.0 g, 4.5 g, 4.0 g, 3.5 g, 3.0 g, 2.5 g, 2.0 g, 1.5 g, or 1.0 g, of pigments per liter of culture. The culturing may generate 0.5 g to 7.0 g, 1 g to 6.0 g, 2 g to 5.0 g, 3.0 g to 4.5 g, or 3.5 g to 4.0 g of pigments per liter of culture. The culturing may generate about 1.0 to 3.0 grams of the monascorubraminic acid pigments per liter of culture.

Methods of producing colorant compositions disclosed herein may produce at least about 0.5 g, 1.0 g, 1.5 g, 2.0 g, 2.5 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, or 10.0 g of recovered colorant composition per liter of liquid culture, or a range between any two of these values (e.g., 3.0 to 8.0, 4.0 to 8.0, 5.0 to 8.0, 6.0 to 8.0, 7.0 to 8.0, 4.0 to 7.5, 5.0 to 7.5, 6.0 to 7.5, etc.). Methods of producing colorant compositions disclosed herein may produce at least about 0.02 g, 0.04 g, 0.06 g, 0.08 g, 0.1 g, 0.11 g, 0.12 g, 0.13 g, 0.14 g, 0.15 g, 0.16 g, 0.18 g, 0.19 g, or 0.20 g of colorant composition per gram of carbon source, or a range between any two of these values, in the liquid growth medium.

Cultures may be evaluated and characterized by the production of biomass or pigment. For example, one parameter that may be used to characterize a culture is the productivity (Qp), which may be expressed in units of grams of pigment produced per liter of broth per hour. In some embodiments, a culture of pigment-producing fungi has a Qp of at least 0.005, 0.01, 0.02, 0.03, 0.04, 0.05, 0.06, 0.08, 0.09, 0.10, 0.11, 0.12, 0.13, 0.14, or 0.15 grams of pigment produced per liter of broth per hour, or a range between any two of these values. The titer of pigment in a culture can be calculated by, for example, measuring absorbance at 505 nm and using the equation: (0.0346×absorbance)−0.0000087.

Another parameter that may be used to characterize a culture is specific productivity (qp), which may be expressed in units of grams of colorant produced per gram of biomass (dry weight) per hour. Dry weight of fungal biomass may be measured, for example, by filtering a known volume of culture with cellulose paper with a suitable pore size (e.g., 30-40 μm), drying the filter paper, weighing the dried filter paper, and comparing the weight of the dried filter paper to the pre-filter weight of the filter paper. In some embodiments, a culture of pigment-producing fungi has a specific productivity (qp) of at least 0.0001, 0.0002, 0.0003, 0.0004, 0.0005, 0.0006, 0.0007, 0.0008, 0.0009, 0.001, 0.0011, 0.0012, 0.0013, 0.0014, 0.0015, 0.0016, 0.0017, 0.0018, 0.0019, 0.002, 0.0021, 0.0022, 0.0023, 0.0024, 0.0025, 0.0026, 0.0027, 0.0028, 0.0029, or 0.003, 0.0035, 0.004, 0.005, 0.006, 0.007, 0.008, 0.009, 0.010, 0.015, 0.020, 0.025, or 0.030 grams of colorant produced per gram of biomass (dry weight) per hour, or a range between any two of these values (e.g., 0.001 to 0.02, 0.0015 to 0.02, 0.002 to 0.01 grams of colorant produced per gram of biomass (dry weight) per hour).

Another parameter that may be used to evaluate a culture is the ratio of grams of fungal biomass (dry weight) produced to grams of a carbon source initially provided in a liquid media (Yxs). In some embodiments, a culture of pigment-producing fungi has a ratio of biomass to grams of carbon source of at least 0.05, 0.1, 0.15, 0.2, 0.25, 0.3, 0.35, 0.4, 0.45, 0.5, 0.55, 0.6 grams of biomass (dry weight) produced per gram of carbon source, or a range between any two of these values.

Any aspect of liquid media described herein may be monitored and/or adjusted during or between culturing steps to achieve a desired outcome. A concentration of a component present in the liquid media may be the same or different for liquid media used in different culturing steps. Any aspect of a liquid media embodiment described herein may be combined with any other aspect.

### C. Pigment-Producing Fungi

Culturing methods described herein may be used for production of colorant by, for example, pigment-producing fungi of the genus Talaromyces, Penicillium, or Monascus. In some embodiments, the fungus is of one of the following species: Talaromyces atroroseus, Talaromyces verruculosus, Talaromyces albobiverticillius, Talaromyces purpureogenus, Talaromyces amestolkiae, Talaromyces ruber, Talaromyces flavus, Penicillium purpurogenum, Penicillium oxalicum, Penicillium armenica, Penicillium marneffei, Penicillium atrovenetum, Penicillium rubrum, Monascus purpureus, Monascus ruber, or Monascus pilosus. A person of ordinary skill in the art will recognize that the methods disclosed herein can be used for culturing additional pigment-producing fungi that have similar properties to the genera and species listed above.

Suitable strains of T. atroroseus for use in methods of producing colorant as described herein include, for example, strains 35816 and 1061 from the Agricultural Research Service Culture Collection (US), strain 9777 from the American Type Culture Collection (US), strains 257.37, 364.48, 234.60, 391.96, 113139, 113153, 113154, 124294, 133442, 133443, 133447, 133449, 133450 from the CBS-KNAW Culture Collection (the Netherlands).

### D. Production of Colorant Composition

Cultures of pigment-producing fungi may be used to make a colorant composition by a method that includes recovering pigments produced by the cultured fungi from a liquid culture. Pigments may be present in dissolved form in the liquid culture. Pigments may also be present within cells of the pigment-producing fungi.

An example of a method of recovering pigments from a culture of pigment-producing fungi to produce a colorant composition may include removing solids and biomass from the liquid culture, producing a pigment solution that is free of solids and biomass. Removing solids and biomass can be done, for example, by filtration or centrifugation. Removing the solids and biomass can be done with or without a step of extracting pigments from within the cultured cells. Extraction can involve lysing the cells, thereby releasing pigments into solution. Extraction may also involve adding a solvent, such as ethanol, ethyl acetate, acetone, or hexane, to the liquid culture to solubilize pigment compounds. The pigment solution may include the monascorubraminic acid pigments.

A method of producing a colorant composition may also include precipitating the pigments in the pigment solution by acidification of the pigment solution. In some cases, ethanol, ethyl acetate, acetone, or hexane extraction of the liquid culture or pigment solution is not performed before the acidification. In some embodiments, extraction, including by, for example, ethanol, ethyl acetate, or other solvents, is not performed at all during the process of recovering pigments from a culture. Acidification may be performed by adding an acid to the pigment solution. The acid may be any suitable acid for precipitating pigments from the pigment solution. The acid may be selected from, for example, sulfuric acid, hydrochloric acid, nitric acid, lactic acid, boric acid, carbonic acid, citric acid, oxalic acid, phosphoric acid, and acetic acid, or any combination thereof. Acidification may be performed by adding sulfuric acid to the pigment solution. The concentration of the sulfuric acid added to the pigment solution may be at least about 10%, 33.5%, 62.18%, 77.67%, or 98% w/v sulfuric acid. The concentration of the sulfuric acid may be adjusted accordingly using water.

Acidification may be performed until the pigment solution or liquid culture is at a particular pH. The acid (e.g., 98% sulfuric acid) may be added until the pigment solution or liquid culture has a pH of at least about 1.5, 2.0, 2.5, 3.0, or 3.5. The pH may be adjusted to at most about 3.5, 3.0, 2.5, 2.0, or 1.5. The pH may be adjusted from about 1.5 to 3.5, 2.0 to 3.0, 2.5 to 3.0, or 2.0 to 2.5. The pH may be about 2.5.

The method may also include recovering the pigments to form the colorant composition. Recovering may include centrifuging the pigment solution after the acidification to form a pellet or allowing precipitated pigment to settle in the bottom of a vessel. The acidified pigment solution may be centrifuged at a speed of at least about 1000 rotations per minute (rpm), 2000 rpm, 3000 rpm, 4000 rpm, 5000 rpm, or more. The acidified extract solution may be centrifuged at at most about 5000 rpm, 4000 rpm, 3000 rpm, 2000 rpm, 1000 rpm, or less. The acidified extract solution may be centrifuged from about 2000 rpm to 5000 rpm, 3000 rpm to 5000 rpm, 3000 rpm to 5000 rpm, 4000 rpm to 5000 rpm, 3000 rpm to 4000 rpm. After centrifugation, the pellet may include the pigments, including monascorubraminic acid pigments, and a supernatant. The supernatant may be removed from the pellet. The pellet may be separated from the supernatant by filtering. The supernatant may be separated from the pellet by pouring the supernatant away from the pellet. Recovering precipitated pigments from the acidified pigment solution can also be performed by filtration.

The recovered precipitated pigment, which may be in a centrifugation pellet or filter cake, may be washed with a wash solution. The washing may involve resuspending the precipitated pigment composition in a wash solution and then centrifuging or filtering the precipitate. This process can be repeated multiple times. The solution may completely dissolve the pellet. The solution may partially dissolve the pellet. The solution may be used to resuspend the pellet. The wash solution may include a base dissolved in a protic solvent. The base may be selected from, for example, potassium hydroxide, sodium hydroxide, barium hydroxide, cesium hydroxide, sodium carbonate, sodium bicarbonate, or calcium carbonate, or any other suitable base. The base may be potassium hydroxide. The protic solvent may be selected from, for example, water, ethanol, methanol, isopropanol, acetic acid, formic acid, or n-butanol. The protic solvent may be water. The protic solvent may be ethanol.

The wash solution may include a base (e.g., potassium hydroxide) having at least about 0.01% weight per volume (% w/v), 0.05% w/v, 0.1% w/v, 0.2% w/v, 0.3%, 0.4%, 0.5%, 0.6% w/v, or a range between any two of these values (e.g., 0.05 to 0.5% or 0.3% to 0.5%). The pellet may be treated with a solution with a base having at most about 3.0% w/v, 2.5% w/v, 2.0% w/v, 1.5% w/v, 1.0% w/v, 0.5% w/v, 0.2% w/v, 0.1% w/v, 0.05% w/v, 0.01% w/v. The pellet may be treated with a solution with a base having from about 0.05% w/v to 3.0% w/v, 0.05% w/v to 1.5% w/v, 0.1% w/v to 2.0% w/v, 0.1% w/v to 1.5% w/v, and 0.1% w/v to 1.0% w/v. The solution may include water and about 0.01% w/v to 2.0% w/v of potassium hydroxide. The solution may have about 0.1% w/v to 1.0% w/v of potassium hydroxide. The solution may have about 0.2 to 1.0% w/v of potassium hydroxide. The pellet may be washed with ethanol.

After recovering pigments from the acidified pigment solution, for example, by centrifugation or filtration, the supernatant or filtrate may include additional pigments that were not precipitated by the acidification. The additional pigments may be extracted. The supernatant or filtrate may be acidic. The supernatant or filtrate may have a pH of about 2.5. The pH may be adjusted as described elsewhere herein. The additional monascorubraminic acid pigments may be extracted with a solvent to form an extraction liquid. The solvent may be ethyl acetate. A solution that includes a base may be added to the extraction liquid to form a basified extraction liquid. The basified extraction liquid may include a water portion and a solvent portion. The final volume of the extraction liquid may be a particular ratio of water portion to solvent portion (e.g., ethyl acetate). The final volume ratio may be at least about 0.01, 0.05, 0.075, 0.1, 0.125, 0.15, 0.2, 0.3, 0.4, 0.5, 0.6, 0.7, 0.8, 0.9, or 1.0 or more, or a range between any two of these values (e.g., 0.05 to 0.15). The final volume ratio may be at most about 0.5, 0.4, 0.3, 0.2, 0.15, 0.125, 0.1, 0.075, 0.05, 0.01, or less. The final volume ratio may be from about 0.01 to 0.5, 0.01 to 0.3, 0.01 to 0.2, 0.01, to 0.1, 0.05 to 0.5, 0.05 to 0.3, 0.05 to 0.2, 0.05 to 0.1, 0.05 to 0.2, 0.05 to 0.2, or 0.1 to 0.2.

The aqueous portion of the extraction liquid may be separated from the solvent portion. The aqueous portion of the basified extraction liquid may be separated from the solvent portion by pipetting the aqueous portion or pouring out the aqueous portion from the ethyl acetate phase. The aqueous portion may be separated from the solvent portion using an extraction funnel. The aqueous portion may be dried to remove water to recover the pigments. The water portion may be dried using spray drying. The solvent of the extraction liquid may be evaporated to recover the additional monascorubraminic acid pigments in a dried form. Evaporating may include spray drying. The resulting dried colorant composition may retain some moisture. In some embodiments, the colorant has a moisture content below 15, 14, 13, 12, 11, 10, 9, 8, 7, 6, 5, 4, 3, 2, 1, 0.5, or 0.1%, or has a moisture content in a range of 10 to 15, 5 to 10, 1 to 5, 0.1 to 3, 7 to 13, 8 to 14, or 10 to 14% w/w.

Methods for producing colorant compositions comprising monascorubraminic acid pigments from fungal cultures, as well as properties and makeup of colorant compositions, are further described in pending U.S. provisional application No. 63/257,473, which is hereby incorporated by reference in its entirety for all purposes.

### E. Colorant Compositions

A colorant composition produced by embodiments of methods disclosed herein may be a solid and may include, for example, one or more of the following pigments: N-glutaryl monascorubraminic acid, N-asparagyl monascorubraminic acid, N-aspartyl monascorubraminic acid, N-cysteinyl monascorubraminic acid, N-phenylalanyl monascorubraminic acid, N-lysyl monascorubraminic acid, N-methionyl monascorubraminic acid, N-glutamyl monascorubraminic acid, and N-arginyl monascorubraminic acid.

A colorant composition produced by embodiments of methods disclosed herein may be a solid and may include, for example, cis-N-glutaryl monascorubraminic acid, trans-N-glutaryl monascorubraminic acid, N-glutaryl-monascorubramine, and N-glutamyl monascorubraminic acid. In some embodiments, the colorant compositions provided herein may further comprise ash, and/or succinate.

The colorant compositions provided herein may be affected by the methods used to prepare such compositions, such as the culturing conditions and the extraction conditions. Thus the culturing and extraction conditions may also affect the resulting colorant visual characteristics as described herein.

In some embodiments, the colorant compositions provided herein comprise cis-N-glutaryl monascorubraminic acid, which exhibits a red color. In some embodiments, cis-N-glutaryl monascorubraminic acid is present in the colorant compositions in an amount of about 60% to about 75%. In some embodiments, cis-N-glutaryl monascorubraminic acid is present in the colorant compositions in an amount of at least 55% (e.g., 57%, 60%, 62%, 64%, 66%, 68%, 70%, 72%, 74%, or 76%). In some embodiments, cis-N-glutaryl monascorubraminic acid is present in the colorant compositions in an amount of at most 80%% (e.g., 78%, 76%, 74%, 72%, 70%, 68%, 66%, or 64%). In some embodiments, cis-N-glutaryl monascorubraminic acid is present in the colorant compositions in an amount of about 55% to about 78%, about 60% to about 76%, about 62% to about 70%, or about 62% to about 68%. In some embodiments, cis-N-glutaryl monascorubraminic acid is the principal pigment in the colorant compositions. In specific embodiments, cis-N-glutaryl monascorubraminic acid is present in the colorant compositions in an amount of about 65%%. In some embodiments, cis-N-glutaryl monascorubraminic acid is present in the colorant compositions in an amount of about 60% to about 75%. In some embodiments, cis-N-glutaryl monascorubraminic acid is present in the colorant compositions in an amount of about 60% to about 70%.

In some embodiments, the colorant compositions provided herein comprise trans-N-glutaryl monascorubraminic acid, which exhibits a purple color. In some embodiments, trans-N-glutaryl monascorubraminic acid is present in the colorant compositions in an amount of at least 1% (e.g., 2%, 3%, 4%, 5%, 6%). In some embodiments, trans-N-glutaryl monascorubraminic acid is present in the colorant compositions in an amount of at most 10% (e.g., 9%, 8%, 7%, 6%, 5%, 4%, 3%). In some embodiments, trans-N-glutaryl monascorubraminic acid is present in the colorant compositions in an amount of about 1% to about 7%, about 2% to about 6%, about 2% to about 5%, or about 2% to about 4%. In some embodiments, trans-N-glutaryl monascorubraminic acid is present in the colorant compositions in an amount of about 3%. In some embodiments, trans-N-glutaryl monascorubraminic acid is present in the colorant compositions in an amount of about 1% to about 7%.

In some instances, the cis- and trans-isomers of N-glutaryl monascaorubraminic acid can be identified by nuclear magnetic resonance, such as described in Example 11.

In some embodiments, a ratio of cis-N-glutaryl monascorubraminic acid to trans-N-glutaryl monascorubraminic acid in a colorant composition herein is about 18:1 to about 25:1. In some embodiments, the ratio of cis-N-glutaryl monascorubraminic acid to trans-N-glutaryl monascorubraminic acid in a colorant composition herein is about 19:1, 20:1, 21:1, 22:1, 23:1, or 24:1. In some embodiments, the ratio of cis-N-glutaryl monascorubraminic acid to trans-N-glutaryl monascorubraminic acid in a colorant composition herein is about 22:1.

In some embodiments, the colorant compositions provided herein comprise N-glutamyl monascorubraminic acid. In some embodiments, the colorant compositions herein comprise N-glutamyl monascorubraminic acid in an amount of at least 1% (e.g., 2%, 3%, 4%, 5%, 6%). In some embodiments, the colorant composition herein comprise N-glutamyl monascorubraminic acid in an amount of at most 10% (e.g., 9%, 8%, 7%, 6%, 5%, 4%, 3%). In some embodiments, the colorant compositions herein comprise N-glutamyl monascorubraminic acid in an amount of about 1% to about 7%, about 2% to about 6%, about 2% to about 5%, or about 2% to about 4%. In some embodiments, the colorant compositions herein comprise N-glutamyl monascorubraminic acid in an amount of about 3%. In some embodiments, the colorant compositions herein comprise N-glutamyl monascorubraminic acid in an amount of about 2% to about 6%.

In some embodiments, the colorant compositions herein comprise N-glutaryl monascorubramine. In some embodiments, the colorant compositions herein comprise N-glutaryl monascorubramine in an amount of at least 0.5% (e.g., 1%, 1.5%, 2%, 2.5%, 3%, or 4%). In some embodiments, the colorant compositions herein comprise N-glutaryl monascorubramine in an amount of at most 5% (e.g., 4.5%, 4%, 3.5%, 3%, 2.5%, 2%, or 1%). In some embodiments, the colorant compositions provided herein comprise N-glutaryl monascorubramine in an amount of about 0.5% to about 5%, about 1% to about 3%, about 1% to about 2%, or about 1% to about 2%. In some embodiments, the colorant compositions provided herein comprise N-glutaryl monascorubramine in an amount of about 2% (e.g., 1.8%). In some embodiments, the colorant compositions provided herein comprise N-glutaryl monascorubramine in an amount of about 1% to about 4%.

In some embodiments, the colorant compositions herein comprise ash. In some instances, ash comprise metals from the culture medium. In some embodiments, the colorant compositions comprise ash in an amount of at least 1% (e.g., 2%, 3%, 4%, 5%, or 6%). In some embodiments, the colorant compositions comprise ash in an amount of at most 6% (e.g., 5%, 4%, 3%, or 2%). In some embodiments, the colorant compositions comprise ash in an amount of about 1% to about 6%, about 1% to about 5%, or about 1% to about 4%. In some embodiments, the colorant compositions comprise ash in an amount of about 3%.

In some embodiments, the colorant compositions comprise succinate. In some embodiments, the colorant compositions comprise succinate in an amount of at least 1% (e.g., 2%, 3%, 4%, 5%, 6%). In some embodiments, the colorant compositions comprise succinate in an amount of at most 15% (e.g., 13%, 11%, 10%, 9%, 8%, 7%). In some embodiments, the colorant compositions comprise succinate in an amount of about 1% to about 15%, about 1% to about 10%, about 2% to about 10%, about 4% to about 9%, or about 5% to about 8%. In some embodiments, the colorant compositions comprise succinate in an amount of about 2% to about 12%. In some embodiments, the colorant compositions comprise succinate in an amount of about 6.5%.

In some embodiments, the colorant compositions can comprise no more than 30% of other compounds. Other compounds as provided herein may comprise, for example, monascorubramine. In some embodiments, other compounds as provided herein comprise, for example, pyruvate. In some embodiments, other compounds may be present in the colorant compositions in an amount of no more than 30% (e.g., 25%, 20%, 15%, 10%, 9%, 8%, 7%, 6%, 5%, 4%, 3%, 2%, or 1%). In some embodiments, other compounds may be present in the colorant compositions in an amount of at least 1% (e.g., 2%, 3%, 5%, 10%, 15%, 20%, or 30%). In some embodiments, other compounds may be present in the colorant compositions in an amount of about 1% to about 30%, about 1% to about 20%, about 1% to about 10%, about 5% to about 25%, about 5% to about 15%, or about 10% to about 30%. In some embodiments, other compounds are present in the colorant compositions in an amount of about 1% to about 10%.

In some embodiments, the colorant compositions herein may comprise 3% ash, 6.5% succinate, 3.2% N-glutamyl monascorubraminic acid, 65.4% cis-N-glutaryl monascorubraminic acid, 3.1% trans-N-glutaryl monascorubraminic acid, and 1.8% N-glutaryl monascorubramine.

In some instances, the percentage (%) of components in the colorant compositions provided herein refers to wt % in relation to the colorant composition as whole, mol % in relation to the colorant composition as a whole, mol % in relation to other pigments present in the colorant composition, or wt % in relation to other pigments present in the colorant composition. In some instances, the (%) of components in the colorant compositions provided herein refers to wt % in relation to the colorant composition as a whole. In some instances the percentage (%) of components in the colorant compositions provided herein refers to the percentage of integrated area in an e.g., chromatogram in relation to the total integrated area (e.g., such as measured by HPLC).

The colorant compositions herein may be characterized (e.g., for their content) using high performance liquid chromatography, mass spectrometry, or a combination thereof, such as described in Examples 12 and 13 herein.

The colorant composition may be solid. The solid colorant composition may have a moisture content below 14, 13, 12, 11, 10, 9, 8, 7, 6, 5, 4, 3, 2, 1, or 0.1%. The colorant composition may be liquid. The colorant composition may be a suspension.

The colorant compositions herein may have a particular color as expressed in a CIELAB color space. The CIELAB color space may be as defined by the 1976 International Commission on Illumination (abbreviated CIE). The CIELAB color space may be also known as CIE L* a* b*. The CIE L* a* b* color space represents quantitative relationship of colors on three axes: L* value indicates lightness, and a* and b* are chromaticity coordinates. On the color space diagram, L* is represented on a vertical axis with values from 0 (black) to 100 (white). The a* value indicates red-green component of a color, where +a* (positive) and −a* (negative) indicate red and green values, respectively. The yellow and blue components are represented on the b* axis as +b* (positive) and −b* (negative) values, respectively. The center of the plane is considered neutral or achromatic. The distance from the central axis represents the chroma (C), or saturation of the color. The angle on the chromaticity axes represents the hue (ω).

CIELAB parameters for colorants disclosed herein can be measured by the following procedure: a solution of the red colorant is prepared by diluting the liquid colorant in 50 mM dipotassium phosphate buffer having a pH in the range of 3.0-9.0, 4.0-7.0, 5.0-6.0, or, preferably 5.0 in order to obtain a solution with an absorbance in the range of 0.3-0.7, 0.3-0.6, or, preferably 0.5. The CIELAB parameters of said solution can be measured using a CIELAB colorimeter that automatically calculates L*, a*, and b*. Alternatively, other color models and spaces can be used, such as, for example, RGB, CMYK, HEX, XYZ, or ACES, and converted to CIELAB parameters according to techniques known in the art. Alternatively, the absorbance spectrum can be determined in the range of 300 nm-700 nm and converted to CIELAB parameters according to techniques known in the art. The illuminants used can be selected from, for example, A, C, D50, D55, D65, and D75. The measuring geometry can be set to 0° or 45° and the observer angle can be set to 2° or 10°. The chroma value (C) can be calculated from a* and b* using the equation [(a*)²+(b*)²]v². The hue value (ω) can be calculated from a* and b* using the equation tan⁻¹(a*/b*).

In some embodiments, the colorant compositions described herein may have a hue having an ω angle measured in the CIELAB color space in the range of about 30° to 40°. The hue may be at least about 30°, 31°, 32°, 33°, 34°, 35°, 36°, 37°, 38°, 39°, 400 or more. The hue may be at most about 40°, 39°, 38°, 37°, 36°, 35°, 34°, 33°, 32°, 31°, or 30° or less. The hue may be from about 30° to 40°, 30° to 38°, 30° to 36°, 30° to 34°, 30° to 32°, 32° to 40°, 32° to 38°, 32° to 36°, 32° to 34°, 34° to 40°, 34° to 38°, or 34° to 36°. In some embodiments, the colorant composition may comprise a hue of about 30° to about 35°. In some embodiments, the colorant composition may comprise a hue of about 34°.

In some embodiments, the colorant composition may have a brightness value (L*) as measured in the CIELAB color scale in the range of about 75 to about 85. In some embodiments, the L* value may be about 73 to about 83. In some embodiments, the L* value may be at least about 75, about 76, about 77, about 78, about 79, about 80, about 81, about 82, about 83, or about 84. In some embodiments, the L* value may be at most about 85, about 84, about 83, about 82, about 81, about 80, about 79, about 78, about 77, about 76, or about 75. In some embodiments, the L* value may be about 75 to 85, about 75 to 84, about 75 to 83, about 75 to 80, about 76 to 80, about 76 to 79, about 77 to 80, or about 77 to 79. In some embodiments, the L* value of a colorant composition provided herein is about 79. In some embodiments, the L* value of a colorant composition provided herein is about 78. In some embodiments, the L* value of a colorant composition provided herein is about 76 to about 80.

In some embodiments, the colorant composition may have an a* value measured in the CIELAB color scale in the range of about 15 to about 25. The a* value may be at least about 13, 14, 15, 16, 17, 18, 19, 20, 21, 22, 23, 24, 25, or more. The a* value may be at most about 25, 24, 23, 22, 21, 20, 19, 18, 17, 16, 15, or less. The a* value may be from about 13 to 25, 15 to 25, 17 to 25, 19 to 25, 20 to 25, 21 to 25, 22 to 25, 23 to 25, or 24 to 25. In some embodiments, the a* value of a colorant composition provided herein is about 22. In some embodiments, the a* value of a colorant composition provided herein is about 19 to about 23.

In some embodiments, the colorant composition may have a b* value measured in the CIELAB color scale in the range of about 20 to about 30. The b* value may be at least about 20, 21, 22, 23, 24, 25, 26, 27, 28, 29, 30 or more. The b* value may be at most about 30, 29, 28, 27, 26, 25, 24, 23, 22, 21, 20, or less. The b* value may be from about 20 to 30, 20 to 28, 20 to 26, 20 to 24, 20 to 22, 22 to 30, 22 to 28, 22 to 26, 22 to 24, 24 to 30, 24 to 28, or 24 to 26. In some embodiments, a colorant composition provided herein has a b* value of about 26. In some embodiments, a colorant composition provided herein has a b* value of about 25. In some embodiments, a colorant composition provided herein has a b* value of about 23 to about 26.

In some embodiments, the colorant composition may have a chroma value (C) measured in the CIELAB color scale in the range of about 45 to about 55. The C value may be at least about 45, 46, 47, 48, 49, 50, 51, 52, 53, or 54, or more. The C value may be at most about 55, 54, 53, 52, 51, 50, 49, 48, 47, or 46, or less. The C value may be from about 45 to about 55, about 45 to about 52, about 46 to about 52, about 46 to about 50, about 48 to about 52, or about 48 to about 55. In some embodiments, the C value of a colorant composition provided herein may be about 50. In some embodiments, the C value of a colorant composition provided herein may be about 48 to about 52.

In some embodiments, the colorant composition may have a hue having an ω angle measured in the CIELAB color scale of from 45° to 55°, a L* value of about 75 to about 85, an a* value of about 15 to about 25, a b* value of about 20 to about 30, and a C value of about 30 to about 40. In some embodiments, the colorant composition further comprises a coloring capacity of about 250 (e.g., or greater).

In some embodiments, the colorant composition provided herein comprise a coloring capacity of at least 200. In some embodiments, the colorant composition provided herein comprises a coloring capacity of at least 220. In some embodiments, the colorant composition comprises a coloring capacity of at least 180 (e.g., at least 200, 210, 220, 230, 240 or 250). In some embodiments, the colorant compositions herein comprise a coloring capacity of at most 280 (e.g., at most 270, 260, 250, 240, or 230). In some embodiments, the colorant compositions provided herein comprise a coloring capacity of about 245. In some instances, the colorant compositions herein comprise coloring capacities greater than that of their individual components.

### F. Colorant Products

In some embodiments, colorant compositions disclosed herein may be formulated for use in a food, medicine, cosmetic, or non-food formulation. Colorant compositions can be, for example, included in vitamins, liquid medicines, or pharmaceutical tablets, pills, or capsules.

In some embodiments, the colorant composition may be at least a portion of a food product. The food product may be a dairy product, a beverage, a powder, a gel, a water-in-oil emulsion, or an oil-in-water emulsion. The food product may be, for example, a baked good, baking mix, beverage or beverage mix, breakfast cereal, cheese, condiment, relish, confection, frosting, frozen dairy dessert, gelatin, pudding, filling, grave, sauce, milk product, plant protein product, processed fruit, fruit juice, snack food, meat product, cellular meat product, meat substitute, alternative meat product, or extruded food product. The alternative meat products or meat substitutes may be for example, burgers. The extruded food products may be, for example, snacks. The color composition may be at least partially dissolved in water and then incorporated into the food. The color composition may be at least partially dissolved in oil and then incorporated into the food.

The colorant composition may be at least partially dissolved in water. The colorant composition may be at least partially dissolved in oil.

The colorant composition may be at least a portion of a skin care or cosmetic product. The skin care product may be formulated as a cream, a lotion, an oil, an oil-in-water emulsion, a water-in-oil emulsion, a powder, or a gel. The colorant composition may be at least a portion of a cosmetic composition. The skin care product may be formulated as a cream, a lotion, an oil, an oil-in-water emulsion, a water-in-oil emulsion, a powder, serum, moisturizer, or a gel. The cosmetic may be, for example, a lipstick gel.

The colorant composition may be at least a portion of a non-food product. The product may be, for example, packaging, paper products, threads, or textiles.

Colorant compositions may comprise, in addition to pigment compounds, excipients and fillers. Colorant compositions may include, for example, anti-caking agents, thickeners, and preservatives.

### Examples

### Culture Media

Tables 1 to 5 below show the composition of growth media used in the below Examples.

TABLE 1
EMI culture medium composition

| Compound | g/L |
|---|---|
| carbon source | 40 |
| KH₂PO₄ | 1 |
| KNO₃ | 5 |
| MgSO₄•7H₂O | 5 |
| KCl | 0.5 |
| FeSO₄•7H₂O | 0.01 |
| trace elements (1000 X) | 1 mL/L |

TABLE 2
AMYG culture medium composition

| Compound | g/L |
|---|---|
| starch | 40 |
| monosodium glutamate | 8.35 |
| KH₂PO₄ | 1.0 |
| MgSO₄•7H₂O | 5 |
| KCl | 0.5 |
| FeSO₄•7H₂O | 0.01 |
| trace elements (1000 X) | 1 mL/L |

TABLE 3
AYP culture medium composition

| Compound | g/L |
|---|---|
| starch | 40 |
| monosodium glutamate | 8.35 |
| MgSO₄•7H₂O | 5 |
| KCl | 0.5 |
| FeSO₄•7H₂O | 0.01 |
| trace elements (1000 X) | 1 mL/L |

TABLE 4
MPPY culture medium composition

| Compounds | g/L |
|---|---|
| anhydride glucose | 40 |
| NaNO₃ | 3 |
| yeast extract | 2 |
| MgSO₄•7H₂O | 0.5 |
| KCl | 0.5 |
| FeSO₄•7H₂O | 0.01 |

TABLE 5
Trace solution

| Compound | g/L |
|---|---|
| FeSO₄•7H₂O | 10 |
| Tetrasodium EDTA | 50 |
| ZnSO₄•7H₂O | 15 |
| H₃BO₃ | 10 |
| MnCl₂•4H₂O | 5 |
| CoCl₂•6H₂O | 1.5 |
| CuSO₄•5H₂O | 2 |
| (NH₄)₆Mo₇O₂₄•4H₂O | 1 |

### Example 1: Carbon Source Utilization

Carbon sources were evaluated for their impact on biomass and colorant yield. Two culture systems were prepared in Erlenmeyer flasks containing 50 mL of EMI medium with different carbon sources in each. One system had 40 g/L of glucose and the other 40 g/L of starch. Both systems were inoculated with 5×10⁸ Talaromyces atroroseus conidia and incubated at pH 5.0, at 28 °C, and agitated at 200 rpm in an orbital shaker for 96 and 72 hrs, respectively. After that, absorbance at 505 nm was measured and used to calculate the titer of colorant (grams of colorant produced per liter of broth) using the equation: (0.0346×absorbance)−0.0000087. The final fungal biomass (X) was measured as dry weight by filtering the whole broth with cellulose paper with a pore size of 30-40 μm and drying the filter plus the solids at 120 °C for 5 minutes. The biomass titer (X) was calculated as the ratio between the biomass dry weight and the culture volume. The titer of colorant was used to calculate productivity (Qp, grams of colorant produced per liter of broth in one hour). Results are shown in Table 6.

TABLE 6

| | | time | | | |
|---|---|---|---|---|---|
| system | abs | (hs) | g/L | X (g) | Qp (g/L/h) |
| glucose | 25.1 ± 0.6 | 96 | 0.86 | 8.3 ± 0.4 | 0.0090 ± 0.0004 |
| starch | 32 ± 2 | 72 | 1.1 | 26 ± 2 | 0.015 ± 0.002 |

The results indicated that growth on starch produced more biomass and a higher titer of red colorant as compared to glucose.

### Example 2: Carbon Source Concentration

Biomass growth and colorant production were assessed over a range of carbon source concentrations. The mycelium suspension used in this experiment as inoculum was produced by inoculating 50 mL of MPPY medium in an Erlenmeyer flask with 1×10⁷ conidia and incubating at a temperature of 28 °C, and an agitation speed of 200 rpm in an orbital shaker for 48 hrs. Three culture systems were prepared in Erlenmeyer flasks containing 50 mL of AMYG medium with different concentrations of starch: 20, 40, and 60 g/L. Each system was inoculated with a 10% v/v of Talaromyces atroroseus mycelium suspension and incubated at pH 5.0, at a temperature of 28 °C, and an agitation speed of 200 rpm in an orbital shaker for 48 hrs.

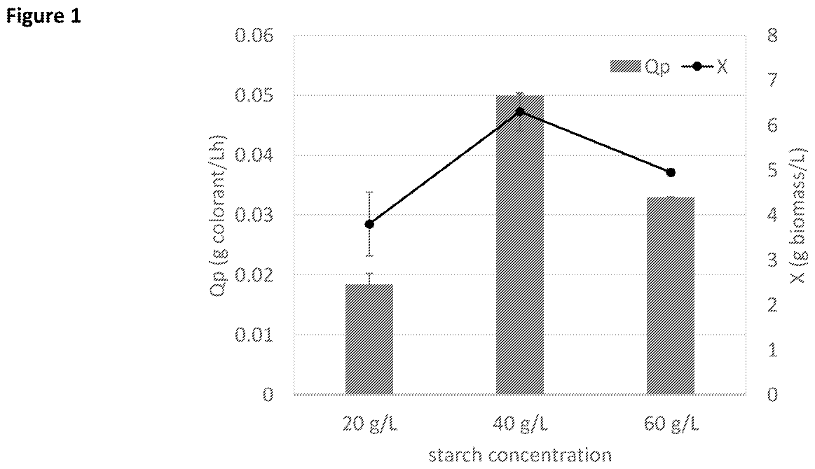

After the growth period, colorant production and biomass were assessed as described in Example 1. The titer of colorant together with the biomass dry weight was used to calculate productivity (Qp, grams of colorant produced per liter of broth in one hour). Also, Yxs was calculated as the ratio between the final fungal biomass and the grams of carbon source. Results are shown in FIG. 1 for Qp and biomass titer (X), respectively. The results indicated that the titer of biomass and colorant was impacted by the concentration of starch, with 20 g/L producing the lowest amount of biomass and colorant, and 40 g/L yielding the highest colorant productivity and biomass production.

### Example 3: Inoculum Evaluation

Conidia and mycelium inoculum (Talaromyces atroroseus) for liquid culture were compared. Two culture systems were prepared in Erlenmeyer flasks containing 50 mL of AMYG medium. One system was inoculated with 2×10⁸ conidia and the other with 10% v/v of mycelium suspension. The mycelium suspension used in this experiment as inoculum was produced by inoculating 50 mL of MPPY medium in an Erlenmeyer flask with 2×10⁸ conidia and incubating at a temperature of 28 °C, and an agitation speed of 200 rpm in an orbital shaker for 48 hrs.

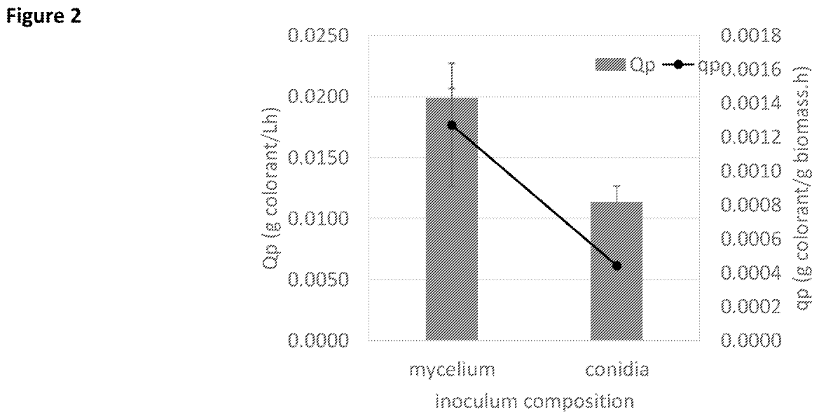

Both systems were incubated at a temperature of 28 °C, and an agitation speed of 200 rpm in an orbital shaker for 48 hrs. Each day, a 5 mL sample was harvested to measure absorbance at 500 nm to track colorant production and dry weight to track biomass growth using the methods described in Example 1 and calculating Qp as described in Examples 1 and 2. Also, the specific productivity (qp, grams of colorant produced by a gram of biomass in one hour) was calculated as the ratio between the colorant titer and the final fungal biomass. Results are shown in FIG. 2. The mycelium inoculum yielded higher productivity and specific productivity. Both produced the same amount of color, but the system inoculated with mycelium reached its maximum in 72 hours, while the system inoculated with conidia reached its maximum in 96 hours.

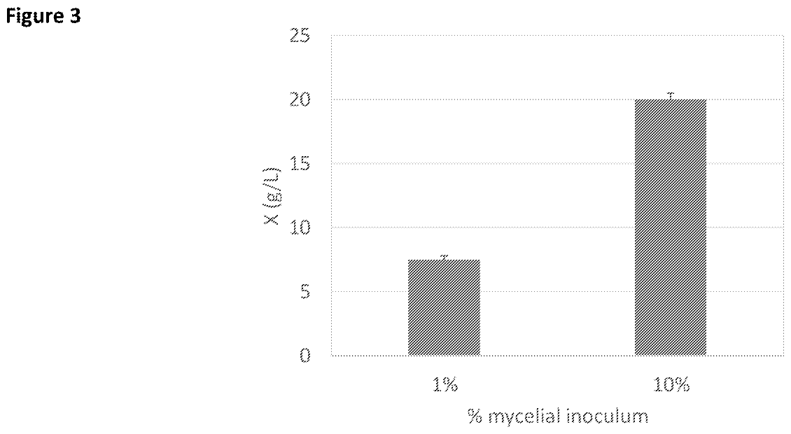

A range of mycelium inoculant was tested for productivity. Two systems were prepared in Erlenmeyer flasks containing 50 mL of MPPY medium. One system was inoculated with 1% v/v of mycelium suspension and the other with 10% v/v of mycelium suspension. Both systems were incubated as described for the conidia/mycelium comparison above and then the final fungal biomass was measured as described in Example 1. Results are shown in FIG. 3. The higher inoculum level (10% v/v) yielded 2.7 times more fungal biomass than 1% v/v.

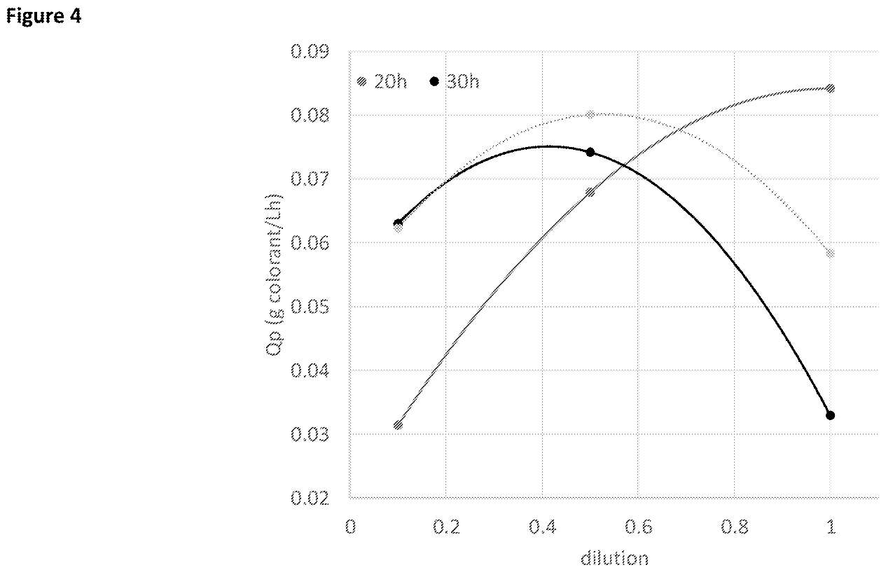

To optimize the size and age of the inoculum, six systems were prepared in Erlenmeyer flasks containing 50 mL of MPPY medium and inoculated with 1×10⁷ conidia. All systems were incubated at 28 °C, and 200 rpm in an orbital shaker for different periods of time: 20, 30, and 40 hrs. Then, samples of these cultures were used to inoculate Erlenmeyer flasks containing 50 mL of AYP medium (10% v/v). Before inoculating the AYP medium, some samples were diluted to 0.5× (1 part culture and 1 part water) and 0.1× (1 part culture and 9 parts water) using distilled water to change the concentration of biomass and simulate a change in the inoculum size. Systems containing AYP medium were inoculated at 28 °C, and 200 rpm in an orbital shaker for 70 hrs. After that, absorbance at 505 nm was measured and used to calculate the titer of colorant (grams of colorant produced per liter of broth) using the following equation (0.0346*absorbance)−0.0000087. The colorant titer was used to calculate productivity (Qp, grams of colorant produced per liter of broth in one hour). FIG. 4 shows that the top 3 age-dilution combinations correspond to:
1. Age=20 h & Dilution=1× (no dilution)
2. Age=40 h & Dilution=0.5×
3. Age=30 h & Dilution=0.5×
   However, both the first and second alternatives result in too much inoculum concentration and too long experimental time, respectively. The third option, on the other hand, results in the best tradeoff between time and concentration, and was thus selected as the most convenient combination. This alternative is expected to lower the costs and efforts of scaling up.

### Example 4: Evaluation of Glutamate as a Nitrogen Source

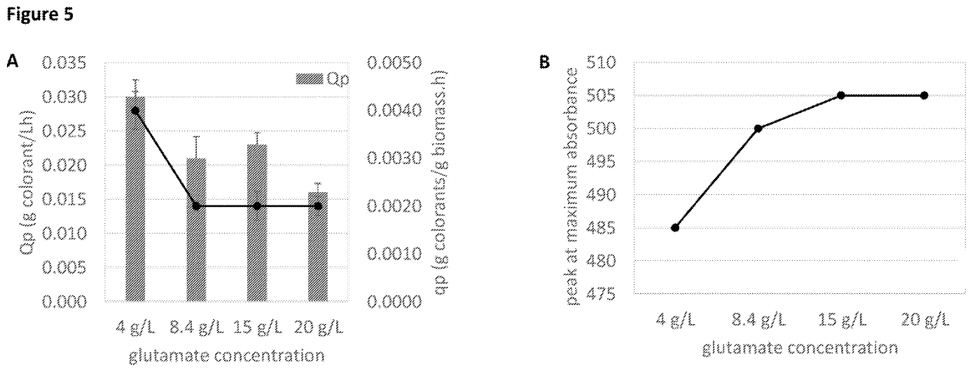

Mycelium suspensions were prepared by inoculating 50 mL of MPPY medium in an Erlenmeyer flask with 1×10⁷ conidia and incubating at pH 5.0, at a temperature of 28 °C and agitated at 200 rpm in an orbital shaker for 48 hrs. Four systems were prepared in Erlenmeyer flasks containing 50 mL of AMYG medium with different concentrations of glutamate: 4.0, 8.4, 15.0, and 20.0 g/L. Each system was inoculated with a 10% v/v mycelium suspension and incubated under the conditions described in Example 2. The titer of colorant and fungal biomass was measured as described in Example 1, with the exception that the wavelength at which the absorbance was measured varies with the concentration of glutamate; the 4 g/L of glutamate produced an orange colorant, with a maximum absorbance at 485 nm, the remaining concentrations of glutamate yielded a red color with a maximum absorbance at 500 and 505 nm (see FIG. 5B). Qp and qp were calculated as described in the preceding examples. Results are shown in FIG. 5A.

### Example 5: Production of Colorant Using Yeast Extract

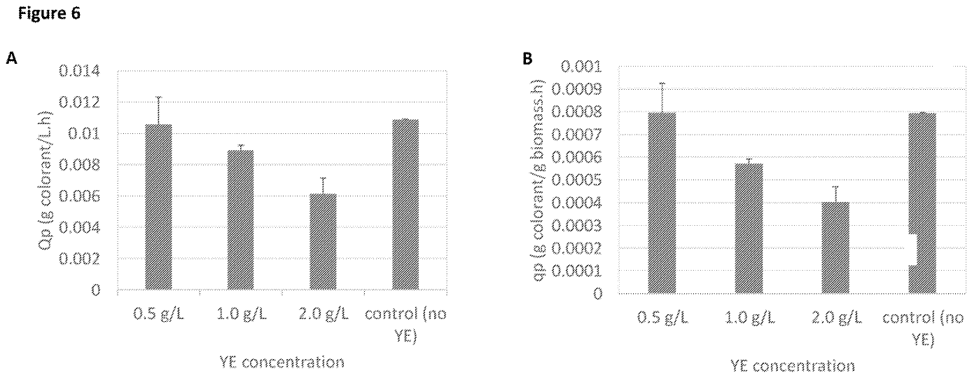

Mycelium inoculum was prepared as described in Example 4. Four culture systems were prepared in Erlenmeyer flasks containing 50 mL of AMYG medium with different concentrations of yeast extract (YE): 0, 0.5, 1.0 and 2.0 g/L. Each system was inoculated with a 10% v/v mycelium suspension and incubated as described in the preceding examples. Titer of colorant, fungal biomass, and calculations of Qp and qp were performed as described in the preceding examples. Results are shown in FIG. 6A for Qp and FIG. 6B for qp. No significant difference in biomass or colorant titer was observed across the yeast extract concentrations tested.

### Example 6: Evaluation of Colorant Production Using Different Concentrations of Phosphate

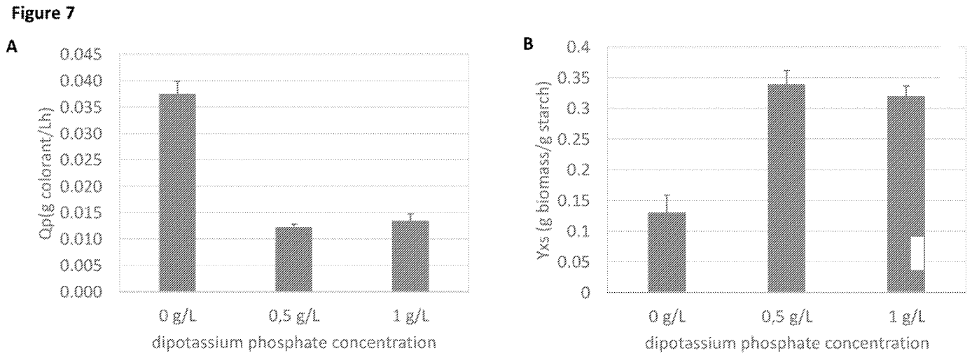

Mycelium inoculum was prepared as described in Example 4. Three culture systems were prepared in Erlenmeyer flasks containing 50 mL of AMYG medium with different concentrations of dipotassium phosphate: 0, 0.5 and 1.0 g/L. Each system was inoculated with a 10% v/v mycelium suspension and incubated as described in Example 2. Qp and Yxs were calculated as described in the preceding examples. Results are shown in FIG. 7A and FIG. 7B. The highest productivity was obtained in absence of phosphate. Although Yxs is the lowest in the absence of phosphate, the productivity was highest.

### Example 7 Production of Colorant Using Yeast Extract in Culture Media without Phosphate

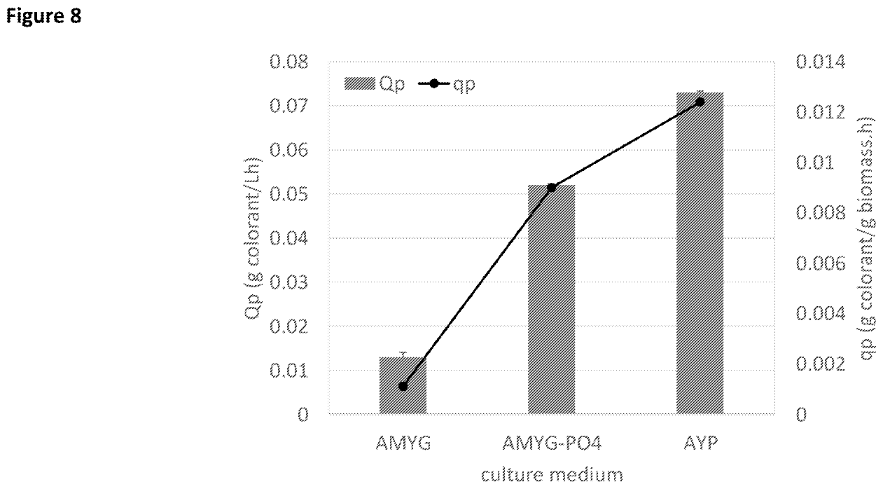

Mycelium inoculum was prepared as described in Example 4. Two systems were prepared in Erlenmeyer flasks containing 50 mL of AMYG medium without dipotassium phosphate (AMY-PO4) and AYP medium (without dipotassium phosphate and with yeast extract). A system prepared with AMYG was used as a control. Each system was inoculated with a 10% v/v mycelium suspension and incubated at Ph 5.0, at a temperature of 28 °C and at an agitation rate of 200 rpm in an orbital shaker for 72 hrs. Qp, qp, and biomass titer were calculated as described in the preceding examples. Results are shown in Table 7 (biomass titer) and in FIG. 8. The elimination of dipotassium phosphate from the culture medium negatively affected the production of biomass but resulted in a higher qp. The addition of yeast extract to a culture medium lacking dipotassium phosphate (AYP) improved both the productivity and specific productivity of the colorant.

TABLE 7

| | Biomass titer, |
|---|---|
| Culture medium | X (g/L) |
| AMYG | 13 ± 2 |
| AMYG w/o | 6.60 ± 0.01 |
| dipotassium phosphate | |
| AYP | 7 ± 1 |

### Example 8: Effect of pH on the Production of Red Colorant

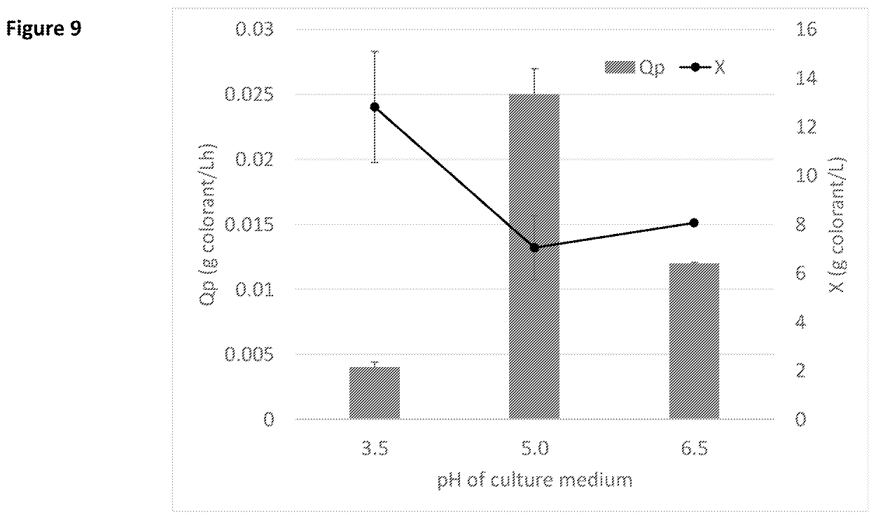

Mycelium inoculum was prepared as described in Example 4. Three systems were prepared in Erlenmeyer flasks containing 50 mL of AMYG medium and incubated at different Ph: 3.5, 5.0, and 6.5. Each system was inoculated with a 10% v/v mycelium suspension and incubated at a temperature of 28 °C and agitated at 200 rpm in an orbital shaker for 48 hrs. Colorant production and biomass were measured as described in Example 1, and Qp and biomass titer (X) were calculated as described in the preceding examples. Results are shown in FIG. 9. Results showed that colorant productivity is highest at pH 5.0 while fungal biomass is highest at pH 3.5.

### Example 9: Effect of Stirring Speed on the Production of Red Colorant

Four systems were prepared in bioreactors containing 2 L of AYP culture medium and inoculated with 10% v/v of a mycelium-based inoculum. The mycelium suspension used in this experiment as inoculum was produced by inoculating 200 mL of MPPY medium in an Erlenmeyer flask with 4×10⁶ conidia and incubating at 28 °C and 200 rpm in an orbital shaker for 48 hrs. The fermentation conditions for the bioreactors were 30 °C, starting pH set to 5.0. The dissolved oxygen was evaluated under two conditions: over 5000 and over 65%, and stirring was evaluated as shown in Table 8.

TABLE 8

| | dissolved | |
|---|---|---|
| system | oxygen | stirring |
| 1 | over 30% | fix at 600 rpm until starch |
| | | depletes, then fix at 800 rpm. |
| 2 | over 50% | fix at 500, 650, or 800 rpm |
| 3 | over 65% | fix at 500, 650, or 800 rpm |

The fermentation process takes 72 hrs and during that time, the absorbance and the biomass concentration (X) are measured to track the production of the colorant. Absorbance at 505 nm was used to calculate the titer of colorant (grams of colorant produced per liter of broth) using the following equation (0.0346*absorbance)−0.0000087. Fungal biomass was measured as dry weight by filtering the whole broth with cellulose paper with a pore size of 30-40 m and drying the filter plus the solids at 120 °C for 5 minutes. The final titer of colorant together with the final biomass dry weight was used to calculate the productivity of the fermentation process (Qp, grams of colorant produced per liter of broth in one hour) and specific productivity (qp, grams of colorant produced by a gram of biomass in one hour), respectively, as shown in Table 9.

TABLE 9

| | | Qp | qp |
|---|---|---|---|
| | stirring | (g colorant/ | (g colorant/ |
| system | (rpm) | L h) | g biomass h) |
| 1 | 600→800 | 0.1027 | 17 |
| 2 | 500 | 0.048 | 9.2 |
| | 650 | 0.044 | 7.3 |
| | 800 | 0.052 | 5.0 |
| 3 | 500 | 0.062 | 7.9 |
| | 650 | 0.058 | 9.4 |
| | 800 | 0.059 | 9.8 |

Results showed that the best stirring strategy is to start at 600 rpm and the switch to 800 rpm when starch depletes.

### Example 10: Optimization of the Culture Medium Composition

An experiment with fractional factorial design was prepared to determine the effect of five culture medium factors on one response. The following factors were evaluated, as described in Table 10 below, and the response variable was Qp, measured in g/Lh.

TABLE 10

| | | |
|---|---|---|
| factor | low | high |

| | | | | |
|---|---|---|---|---|
| glucose:starch ratio | 0:40 | g/L | 10:30 | g/L |
| yeast extract concentration | 0.5 | g/L | 1.0 | g/L |
| Fe₂SO₄ concentration | 0.01 | g/L | 0.04 | g/L |
| KCl concentration | 0.5 | g/L | 1.0 | g/L |
| MgSO₄ concentration | 3.0 | g/L | 5.0 | g/L |

A 2⁵⁻¹ fractional factorial design without replicates was selected as the design to be used on this experiment. As such, it consisted of N=16 total trials. The values on which to fix the factors for each trial were determined so that the design had Resolution V, which avoids main effects and two-way interactions to be confused with one another.

Once the trials were determined, all the systems were prepared in Erlenmeyer flasks containing 50 mL of the specific culture medium at pH 5.0. Each system was inoculated with a 10% v/v mycelium suspension and incubated at 28 °C and 200 rpm in an orbital shaker for 68 hrs. After that, absorbance was measured and used to calculate the titer of colorant (grams of colorant produced per liter of broth) using the equation (0.0346*absorbance)−0.0000087 and used to calculate productivity (Qp, grams of colorant produced per liter of broth in one hour). The mycelium suspension used in this experiment as inoculum was produced by inoculating 50 mL of MPPY medium in an Erlenmeyer flask with 1×10⁷ conidia and incubating at pH 5.0, 28 °C and 200 rpm in an orbital shaker for 48 hrs.

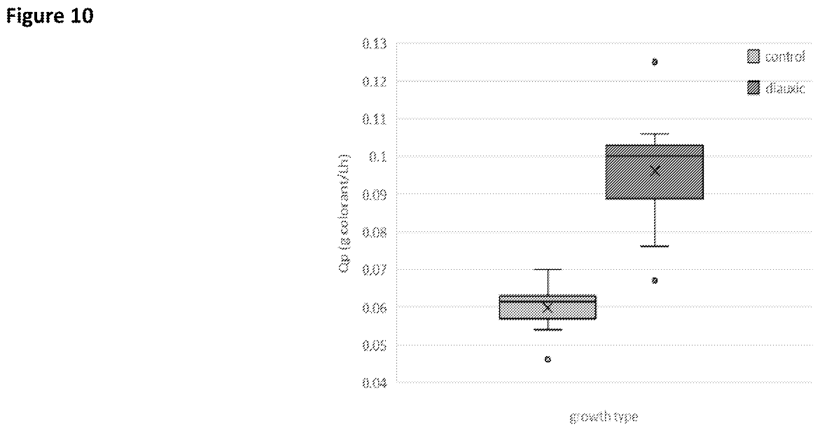

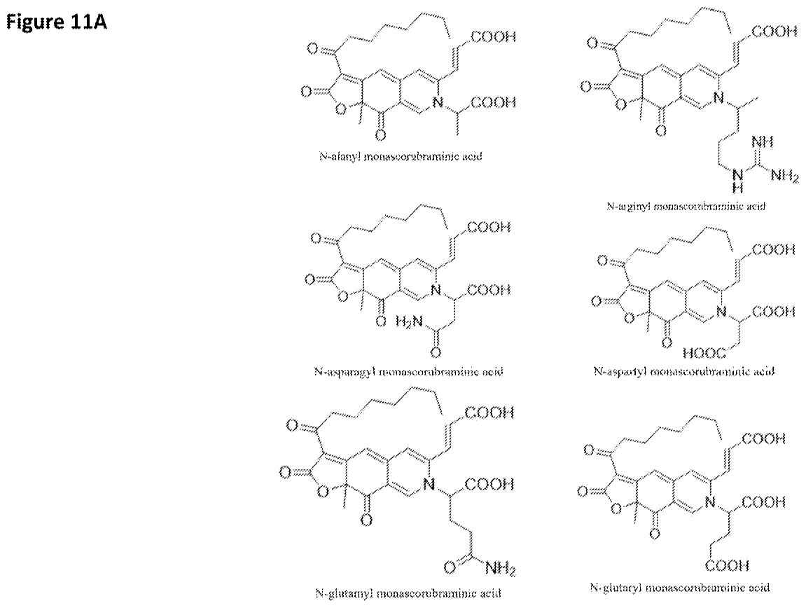

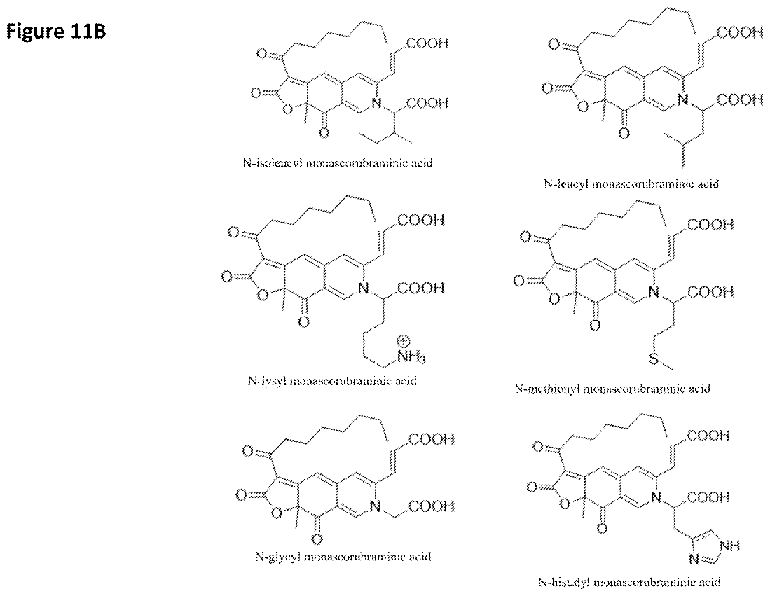

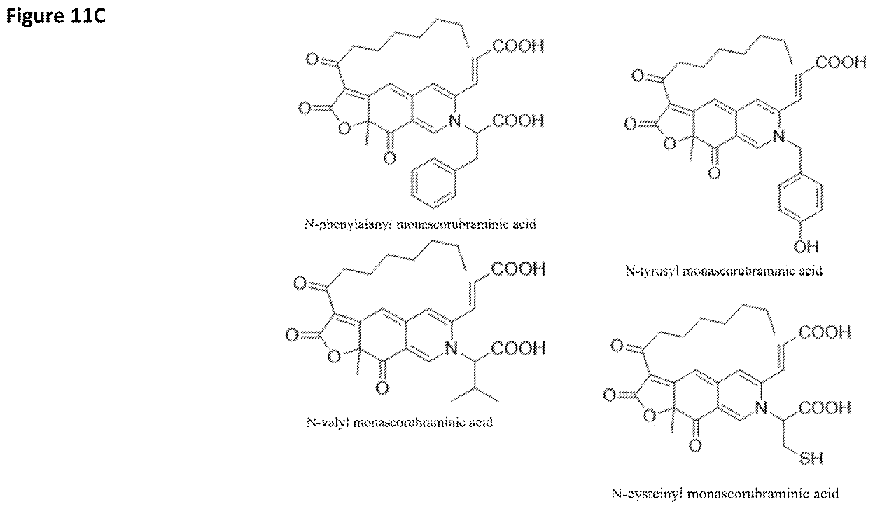

Using the experimental design and the values for the response variable some exploratory data analysis was performed in order to gain general insight into the effect of each factor on the response. For instance, the graph in FIG. 10 shows the distribution of Qp values given the glucose:starch ratio. FIG. 10 shows that the use of 10:30 diauxic systems result in higher values of Qp compared to control (0:40) systems. The statistical significance of said difference was validated through a two-sample Wilcoxon test (p-value<0.001).

A polynomial regression was then performed on the data, being particularly careful in selecting a model as parsimonious as possible while checking all model assumptions. The following formula corresponds to the fitted equation, and results in a value of R2=0.992.

Qp=0.18−0.0044Starch+0.066Y E+1.24Fe₂SO₄+0.105KCl−0.025MgSO₄−0.044Starch*Fe₂SO₄+0.00046Starch*MgSO₄+0.618Y E*Fe₂SO₄−0.083Y E*KCl−1.23Fe₂SO₄*KCl+0.305Fe₂SO₄*MgSO₄

The model was used to find the highest predicted Qp among the conditions set for this experiment. The selected values for the factors under study are shown in Table 11. Its predicted Qp was 0.1221 g/Lh, while its observed Qp was 0.1248 g/Lh.

TABLE 11

| | concentration |
|---|---|
| compound | (g/L) |
| glucose | 10 |
| starch | 30 |
| yeast extract | 1.0 |
| Fe₂SO₄ | 0.04 |
| KCl | 0.5 |
| MgSO₄ | 5.0 |

As the mean Qp for diauxic systems was 60% higher than the mean Qp for control systems, the first preliminary conclusion was to define a new culture medium composition based on diauxic growth. Among the 8 trials contemplating such growth, one of them resulted in a value for Qp equals to 0.1248 g/Lh. This experiment was tested on bioreactors and compared with the previous culture medium composition (AYP). A value for Qp equal to 0.05 g/Lh was obtained using AYP medium, whereas the obtained value using the new culture medium was 0.091 g/Lh. Implementation in bioreactors resulted in an 82% increase in Qp.

### Example 11: Chemical Synthesis

### Synthesis of N-Glutaryl Monascorubramine

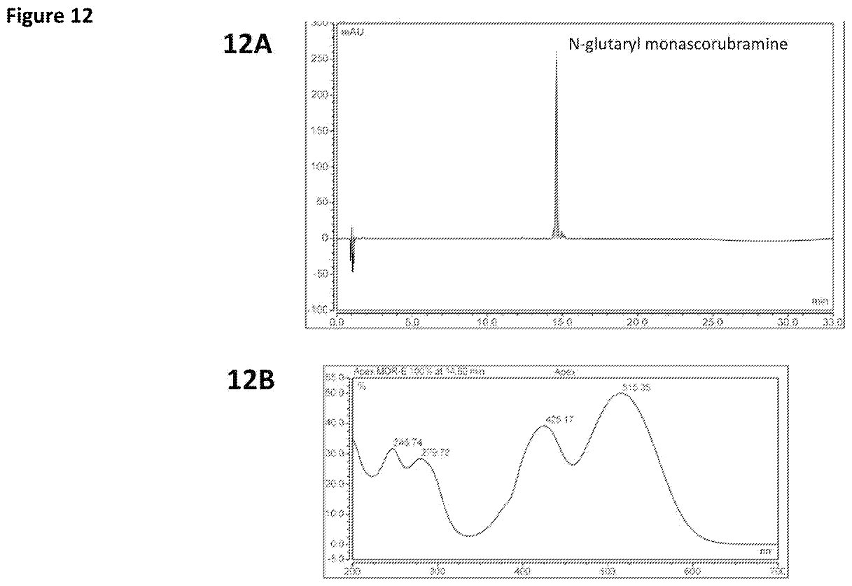

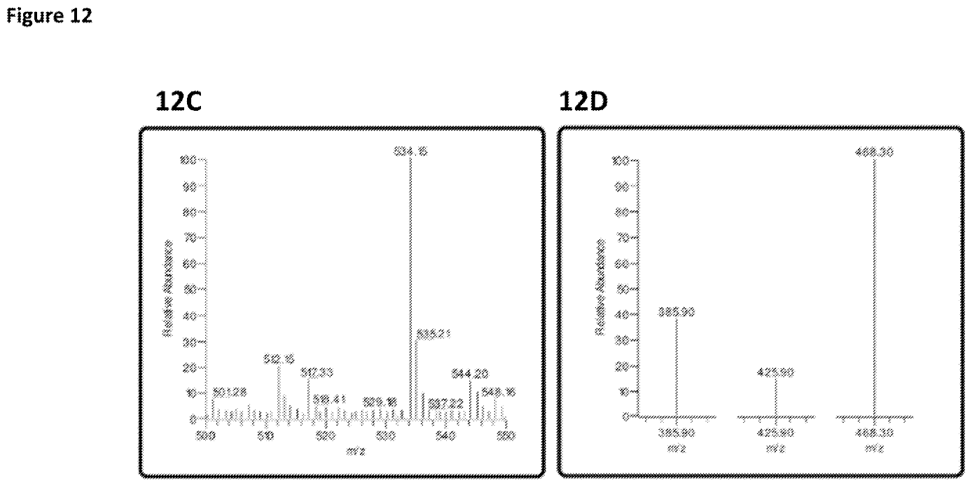

Compound E11-1 (50 mg) is dissolved in 10 mL of ethanol to which 26 mg of monosodium glutamate is added. The system is placed under a nitrogen atmosphere with stirring at room temperature. Finally, 20 microliters of triethylamine are added using a syringe as a reaction catalyst. Upon adding this catalyst, the reaction mixture changes from orange to intense red. The progress of the reaction is monitored by TLC. After 3 hours, to stop the reaction, the system is dried using a rotary evaporator, resulting in an intense red solid. The sample solid is resuspended in methanol and filtered. The reaction crude is analyzed by HPLC. IPLC indicates 70% yield of N-glutaryl monascorubramine. The crude reaction mixture is purified by column chromatography. The IPLC chromatogram and absorbance spectrum of N-glutaryl monascorubramine can be seen in FIG. 12A-B.

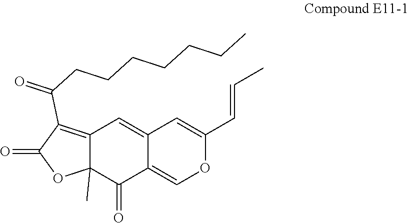 [SMILES: C/C=C/C1=CC2=CC3=C(C(=O)CCCCCCC)C(=O)OC3(C)C(=O)C2=CO1]

### Synthesis of Cis-N-Glutaryl Monascorubraminic Acid & Trans-N-Glutaryl Monascorubraminic Acid

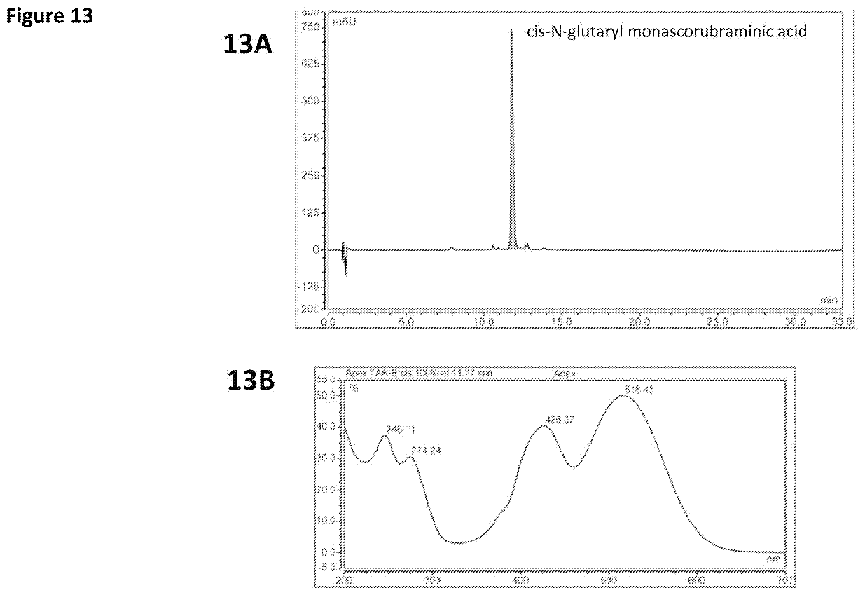

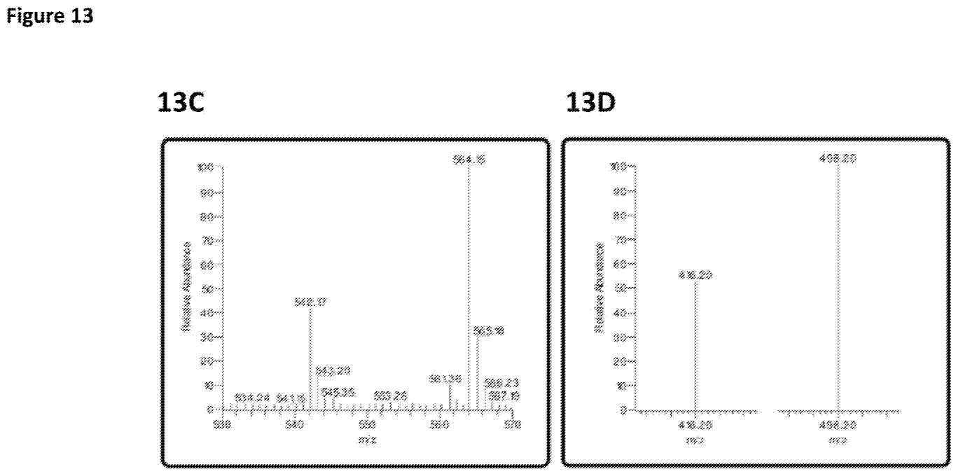

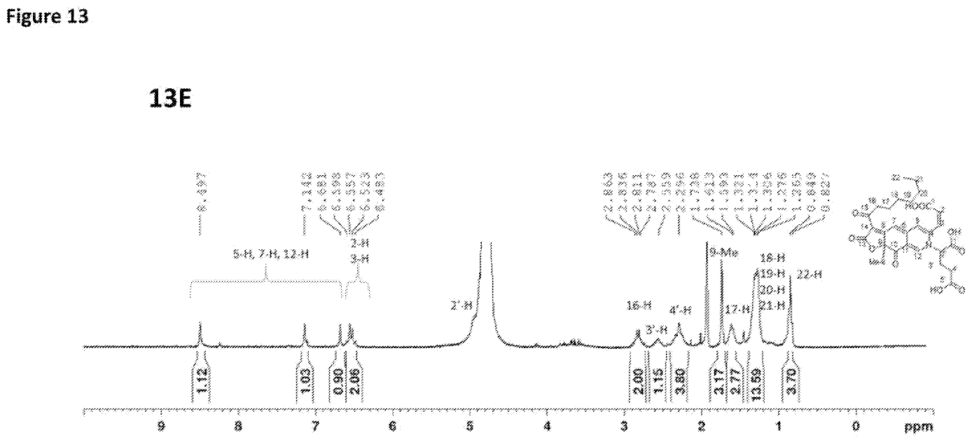

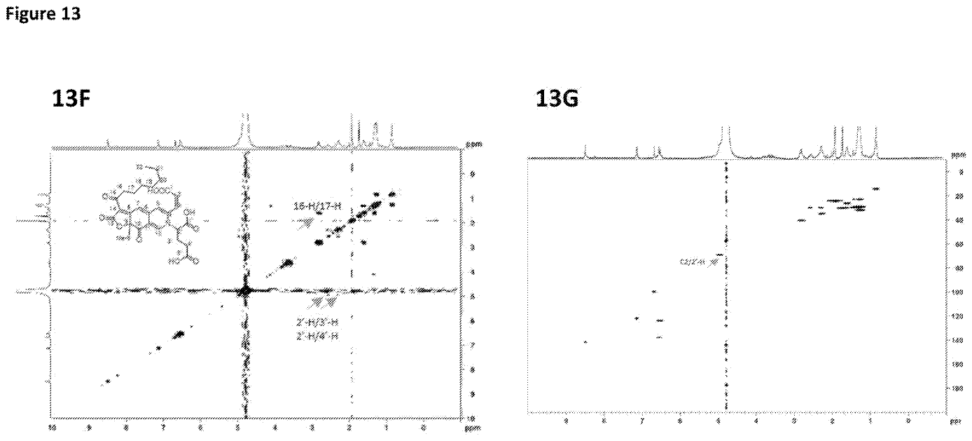

60 mg of Compound E11-2 (below) is dissolved in 10 mL of ethanol to which 22 mg of monosodium glutamate is added. The system is placed under a nitrogen atmosphere with stirring at room temperature. Finally, 20 microliters of triethylamine are added using a syringe as a reaction catalyst. Upon adding this catalyst, the reaction mixture changes from orange to intense red. The progress of the reaction is monitored by TLC. After 3 hours, to stop the reaction, the system is dried using a rotary evaporator, resulting in an intense red solid. The sample solid is resuspended in methanol and filtered. The reaction crude is analyzed by HPLC. IPLC indicates a 77% yield of cis-N-glutaryl monascorubraminic acid. The crude reaction mixture is purified by column chromatography. The IPLC chromatogram and absorbance spectrum of cis-N-glutaryl monascorubraminic acid can be seen in FIG. 13A-B.

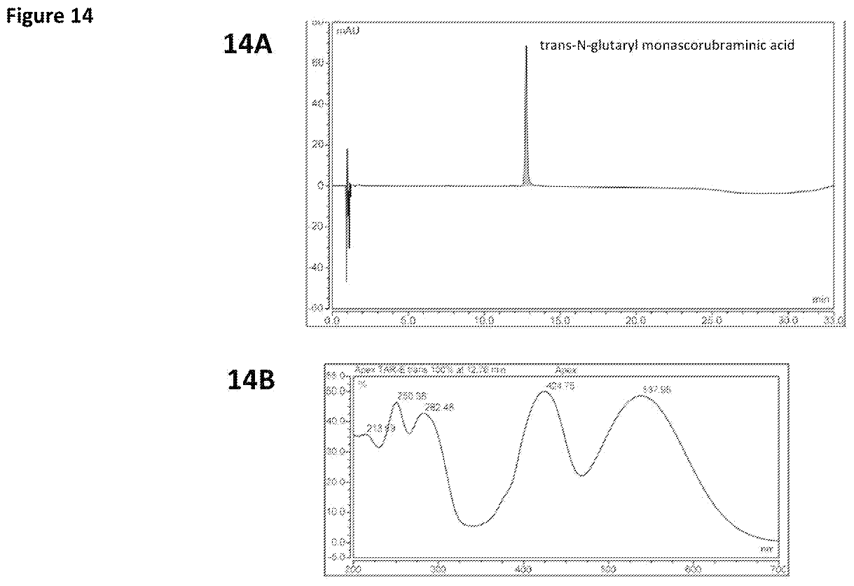

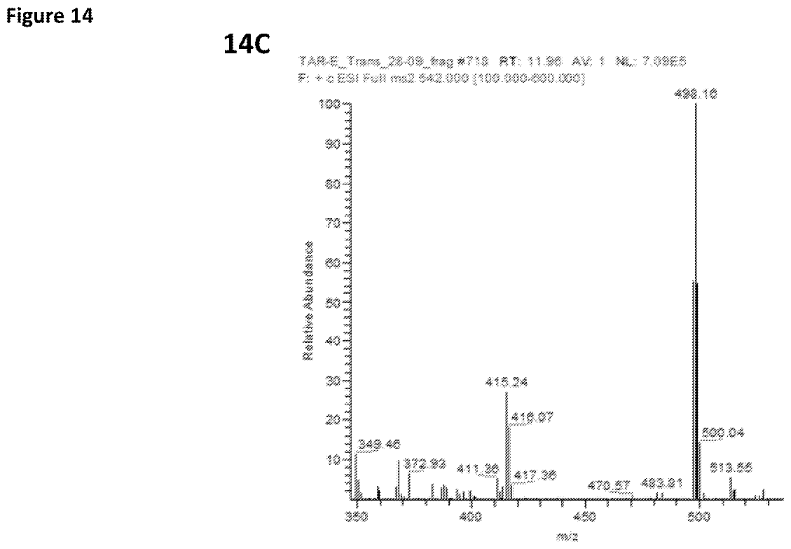

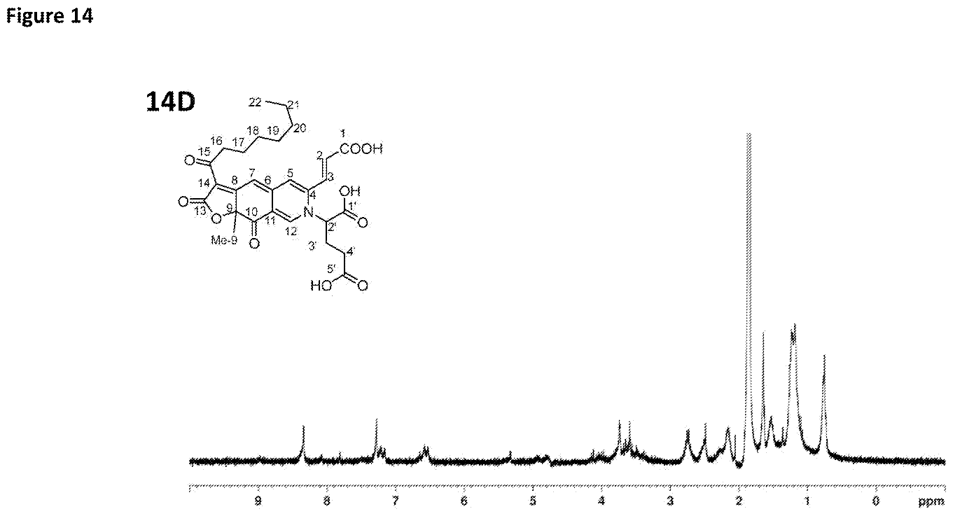

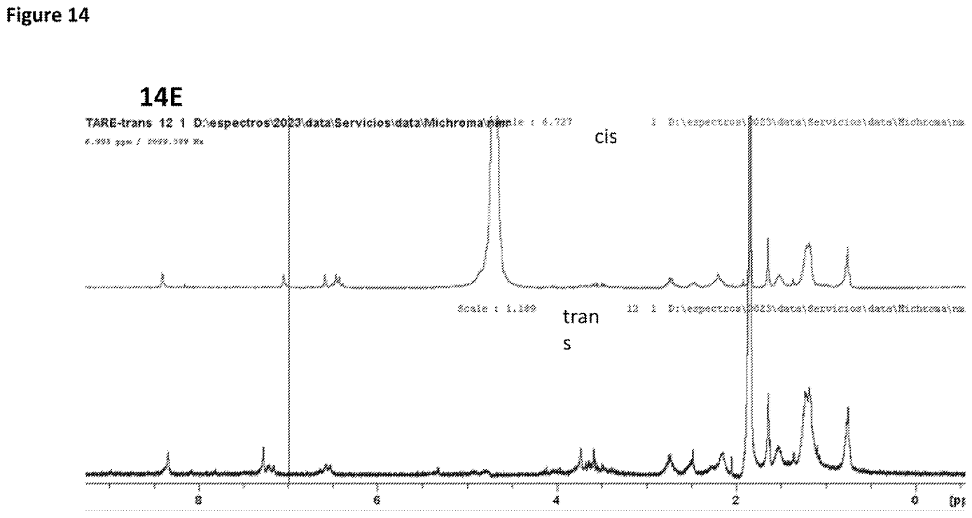

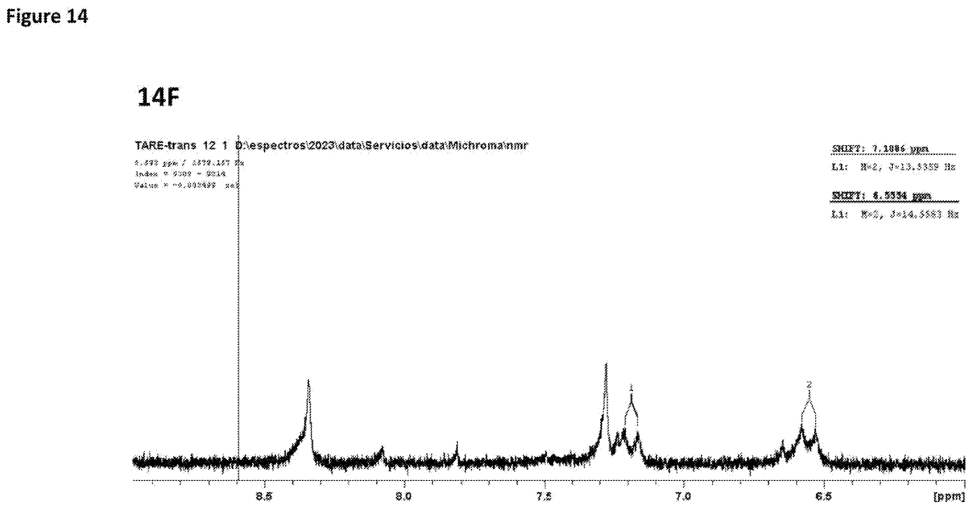

The cis-N-glutaryl monascorubraminic acid compound can be converted to the trans-N-glutaryl monascorubraminic acid by application of UV light for 3 hours. The HPLC chromatogram and absorbance spectrum of trans-N-glutaryl monascorubraminic acid can be seen in FIG. 14A-B.

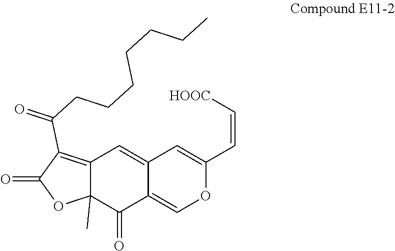 [SMILES: CCCCCCCC(=O)C1=C2C=C3C=C(/C=C\C(=O)O)OC=C3C(=O)C2(C)OC1=O]

### Synthesis of N-Glutamyl Monascorubraminic Acid

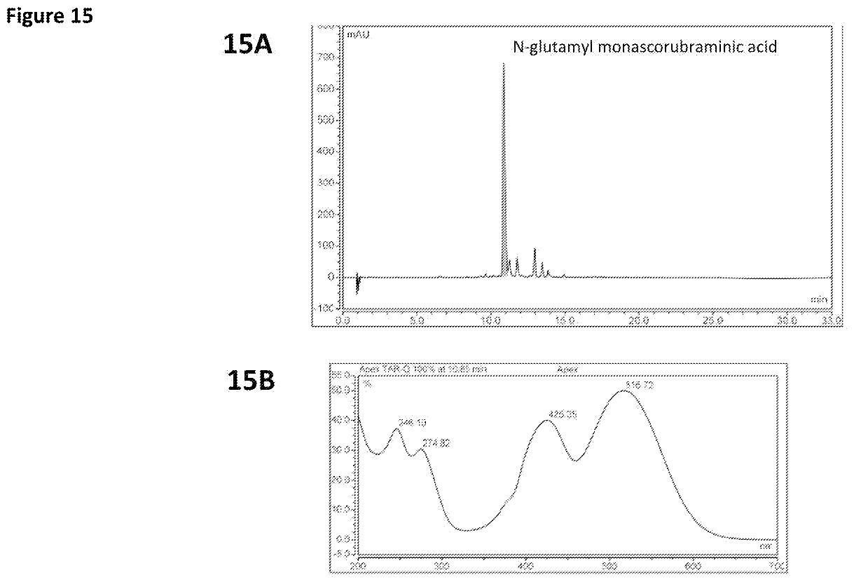

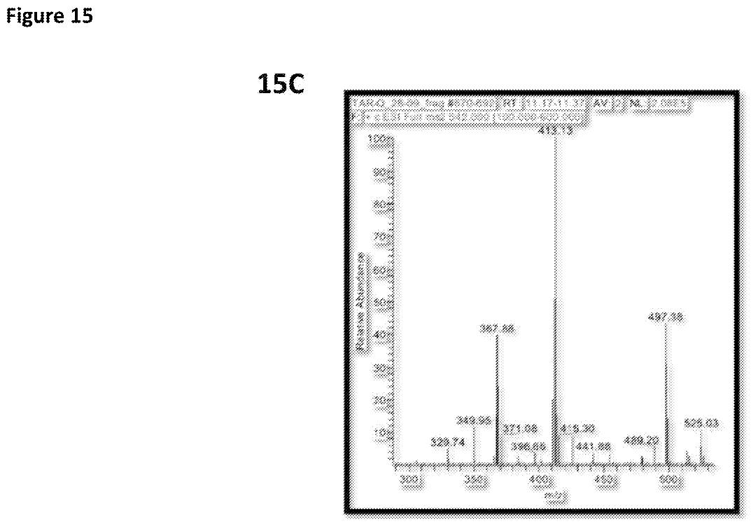

55 mg of Compound E11-2 are dissolved in 10 mL of ethanol to which 19 mg of glutamine are added. The system is placed under a nitrogen atmosphere with stirring at room temperature. Finally, 20 microliters of triethylamine are added using a syringe as a reaction catalyst. Upon adding this catalyst, the reaction mixture changes from orange to intense red. The progress of the reaction is monitored by TLC. After 3 hours, to stop the reaction, the system is dried using a rotary evaporator, resulting in an intense red solid. The sample solid is resuspended in methanol and filtered. The crude reaction mixture is analyzed by HPLC. HPLC indicates 70% yield of N-glutamyl monascorubraminic acid. The crude reaction mixture is purified by column chromatography. The HPLC chromatogram and absorbance spectrum of N-glutamyl monascorubraminic acid can be seen in FIG. 15A-B.

### Example 12: Identification of Components in Pigment Composition

### High Performance Liquid Chromatography

The following protocol describes the methodology for identification, assay and purity evaluation of the red colorant. The cis-N-glutaryl monascorubraminic acid and compounds trans-N-glutaryl monascorubraminic acid, N-glutaryl monascorubramine and N-glutamyl monascorubraminic acid are identified by comparison of the retention times of the peaks in the chromatogram at 520 nm in the Sample Preparation and in the System Suitability Mix. The assay of cis-N-glutaryl monascorubraminic is determined by employing a calibration curve. The purity is determined by means of internal normalization (IN).

The following is completed using a Thermo Scientific Vanquish Core HPLC system consisting of an autosampler organizer, column manager and heater, quaternary pump and PDA absorbance detector. Enterprise Chromeleon 7.3 software is used for system control and data acquisition. The column used is Column Poroshell 120 Phenyl-Hexyl 150 mm×2.1 mm, 2.7 μm. And analytical balance capable of reading to at least 0.1 mg is used. A 0.22 μm pore size membrane filter is used.

Instrumental conditions include: Flow: 0.35 mL/min; Detection: 520 nm; Column temperature: 50 °C; Autosampler temperature: 15 °C; Injection volume: 5 μL (loop volume 20 μL); Run time: 33 minutes; Needle wash solvent: MeOH:H₂O (20:80). The gradient in Table 12 was used.

TABLE 12

| Time | Mobile | Mobile |
|---|---|---|
| (min) | Phase A (%) | Phase B (%) |
| 0 | 80 | 20 |
| 20 | 40 | 60 |
| 23 | 10 | 90 |
| 27 | 10 | 90 |
| 30 | 80 | 20 |
| 33 | 80 | 20 |

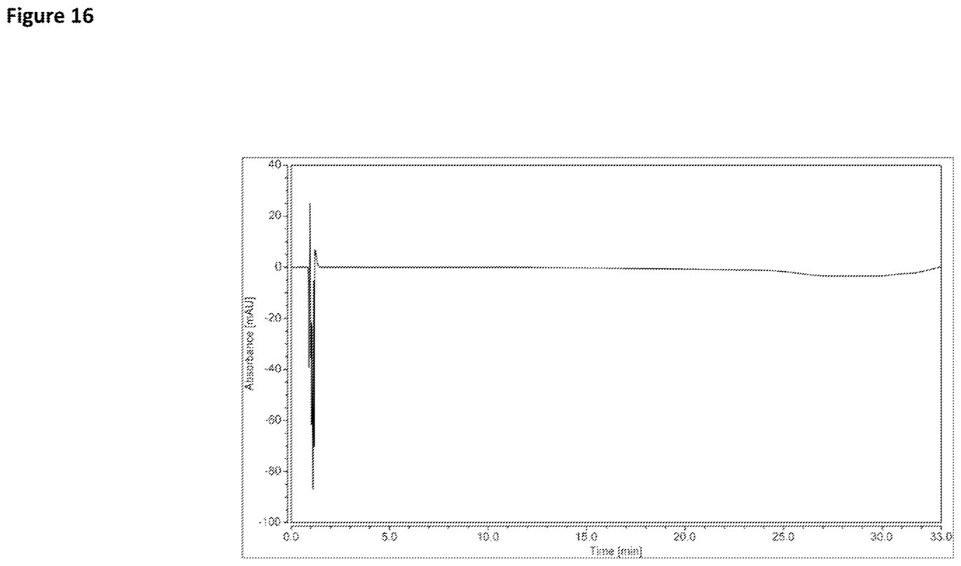

The mobile phase and solvent solution consist of: Mobile Phase A: 0.1% formic acid, filter; Mobile Phase B: 0.1% formic acid in acetonitrile, filter; Solvent solution (SS): MeOH:H₂O (9:1). An HPLC chromatogram of the solvent solution can be seen in FIG. 16.

### REFERENCE AND SAMPLE PREPARATIONS

Stock is prepared (150%) by transferring 15.0 mg of cis-N-glutaryl monascorubraminic acid into a 10 mL volumetric flask and dissolving in the solvent solution. The solution is diluted with the same solvent (Final Concentration=1.5 mg/mL). The calibration curve for N-glutaryl monascorubraminic acid is shown in Table 13 (calculated by the real final concentration of N-glutaryl monascorubraminic acid according to the concentration of Stock corrected by HPLC purity).

TABLE 13

| | | | | Conc N-glutaryl |
|---|---|---|---|---|
| | Aliquot | Vol SS | Vol final | monascorubraminic |
| Level | (mL) | (mL) | (mL) | acid (μg/mL) |
| 1 | 0.10 | 1.40 | 1.5 | 100 |
| 2 | 0.25 | 1.25 | 1.5 | 250 |
| 3 | 0.50 | 1.00 | 1.5 | 500 |
| 4 | 0.75 | 0.75 | 1.5 | 750 |
| 5 | 1.00 | 0.50 | 1.5 | 1000 |
| 6 | 0.85 | 0.15 | 1.5 | 1250 |
| 7 | 1.00 | — | 1.00 | 1500 |

Samples are prepared by adding about 10.0 mg of sample to a 10 mL volumetric flask and dissolving in Solvent Solution. An ultrasonic bath is used where necessary. The solution was diluted to a final concentration of 1 mg/mL.

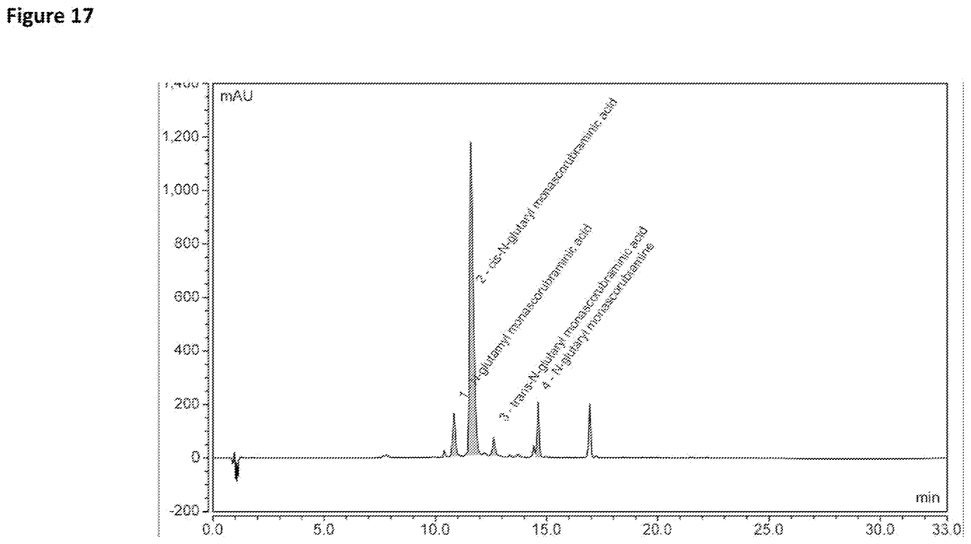

The system suitability mix is prepared with the cis-N-glutaryl monascorubraminic acid, trans-N-glutaryl monascorubraminic acid, N-glutaryl monascorubramine, and N-glutamyl monascorubraminic acidpurified compounds. An HPLC chromatogram of the system suitability mix can be seen in FIG. 17.

### Identification, Assay, and Purity

### Identification of Cis- and Trans-N-Glutaryl Monascorubraminic Acid

The retention time of the cis-N-glutaryl monascorubraminic acid peak in the sample preparation and in the system suitability mix are compared.

### Identification of Known Compounds

Known compounds are identified by comparing the retention times of trans-N-glutaryl monascorubraminic acid, N-glutamyl monascorubraminic acid, and N-glutaryl monascorubramine, in the sample preparation and the system suitability mix. The relative retention times (RRT) of each compound relative to cis-N-glutaryl monascorubraminic acid are detailed in Table 14.

TABLE 14

| Peak | RRT |
|---|---|
| N-glutamyl | 0.94 |
| monascorubraminic | |
| acid | |
| trans-N-glutaryl | 1.09 |
| monascorubraminic | |
| acid | |
| N-glutaryl | 1.25 |
| monascorubramine | |

### Calibration Curve and Content

The calibration curve for the cis and is calculated by integrating the chromatogram peaks and using the below equations. Calibration curves for other components are determined similarly.

The assay (μg/mL) of N-glutaryl monascorubraminic acid (TAR-E) was determined by:

$${C}_{TAR - E ( \frac{ug}{mL} )} = \frac{{A}_{TAR - E} - B}{A}$$

C_{TAR-E} TAR-E concentration (ug/mL)
A_{TAR-E} Area of TAR-E in the Sample Preparation
B Intercept of calibration curve
A Slope of calibration curve

The content (%) of N-glutaryl monascorubraminic acid was determined by:

$${C}_{TAR - E ( \% )} = \frac{{C}_{TAR - E ( \frac{ug}{mL} )} \times {V}_{f} \times 10}{{m}_{SP}}$$

C_{TAR-E} (ug/i) TAR-E concentration (ug/mL)
C_{TAR-E (%)} TAR-E concentration (ug/mL)
Vf Volume of Sample preparation
m_{SP} mass of sample under examination in the Sample Preparation

Table 15 shows the relative areas and retention times for several components of the colorant composition as prepared by methods described herein.

TABLE 15

| | Retention | Relative |
|---|---|---|
| Peak name | Time (min) | Area (%) |
| N-glutamyl | 11.034 | 2.71 |
| monascorubraminic | | |
| acid | | |
| cis N-glutaryl | 11.737 | 76.45 |
| monascorubraminic | | |
| acid | | |
| trans N-glutaryl | 12.774 | 3.70 |
| monascorubraminic | | |
| acid | | |
| N-glutaryl | 14.753 | 1.52 |
| monascorubramine | | |

### Purity of Colorant Composition

The purity of the colorant composition is determined by integrating all peaks in the Sample Preparation. Peaks from the Solvent Solution (if present), were disregarded. The content of each compound was calculated using the following equation (the reporting threshold for compounds is <0.05%).

$$Ci ( \% ) = \frac{{A}_{IC}}{{\sum }_{i = 0}^{i} {A}_{i}}$$

A_{IC} peak area of interest compound in the Sample Preparation

$${\sum }_{i = 0}^{i} {A}_{i}$$

sum of peak area of all compounds in the Sample Preparation

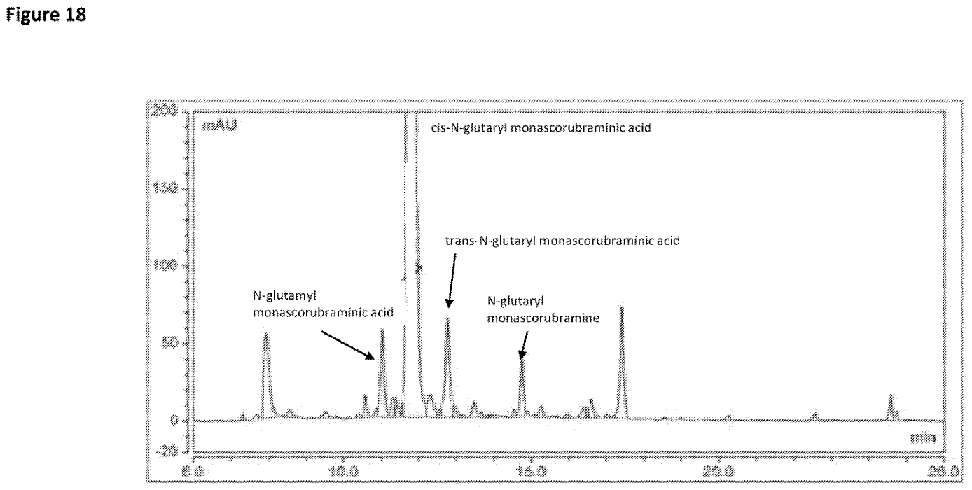

The HPLC chromatogram of a colorant composition as produced by methods described herein can be seen in FIG. 18 where peaks for cis-N-glutaryl monascorubraminic acid, trans-N-glutaryl monascorubraminic acid, N-glutamyl monascorubraminic acid, and monascorubramine are labelled.

### Identification of Compounds in Colorant Composition by Mass Spectrometry

The following protocol describes the methodology for detection and evaluation of colorant composition products by HPLC-UV-MS/MS. The cis-N-glutaryl monascorubraminic acid, trans-N-glutaryl monascorubraminic acid, N-glutaryl monascorubramine, and N-glutamyl monascorubraminic acid components are identified by comparison of the mass spectrometry (MS) spectrum and selected reaction monitoring (SRM) in the Sample Preparation and in the Reference for the known compounds.

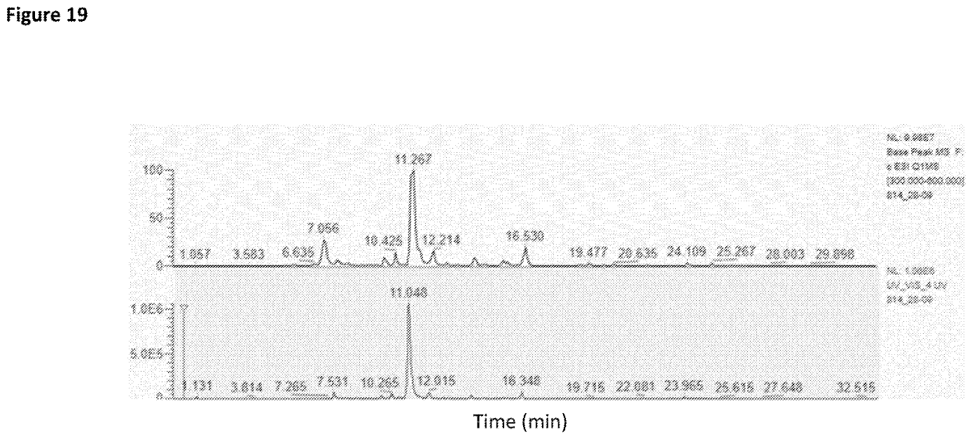

The Base Peak Chromatogram (BPC) is constructed from the base peak abundance of each scan in the analysis, where the base peak in a spectrum is the ion with the maximum abundance, and it shows only the signal from the most intense mass in any given mass spectrum, plotted versus time. The BPC for a colorant composition as prepared by methods described herein is shown in FIG. 19.

The experiments are carried out using a Thermo ScientificUltimate 3000 RSLC, HPLC system consists of an autosampler organizer, column manager and heater, quaternary pump, TSQ Quantum Access Max triple quadrupole mass analyzer and VWD-3400RS detectors. Enterprise Chromeleon 7.3 software is used for data acquisition and system control. The column used is Poroshell 120 Phenyl-Hexyl 150 mm×2.1 mm, 2.7 μm. An analytical balance capable of reading to at least 0.1 mg is used and a membrane filter with 0.22 μm pore size is used.

The instrumental conditions used throughout, unless noted otherwise are: Flow: 0.35 mL/min; Detection Vis: 520 nm; Column temperature: 50 °C; Autosamplertemperature: 15 °C; Injection volume: 5 μL; Run time: 33 minutes; Needle wash solvent: MiOH:H₂O (20:80); Mode: ESC-positive(+); Spray voltage: 3000 V; on transfer capillary temperature: 270 °C; Vaporizer temperature: 220 °C; Sheath gas pressure: 30 psi; Auxiliary gas pressure: 5 psi; Tube lens: +125 UA.

The solvent gradient used is shown in Table 12.

Mobile Phase A is 0.1% formic acid, filter. Mobile Phase B is 0.1% formic acid in acetonitrile, filter. The solvent solution is MeOH:H₂O (9:1). The samples were prepared by transferring 10.0 mg of sample into a 10 mL volumetric flask and dissolving in the solvent solution. An ultrasonic bath was used where necessary. It was diluted to a final concentration of 1 mg/mL using the solvent solution.

The compounds were identified as shown in Table 16.

TABLE 16

| | | Protonated | |
|---|---|---|---|
| | | precursor ion | |
| | | molecular ion | Main ion |
| | Retention | [M + H]⁺ | fragments - MS² |
| Compound | time (min) | (m/z) | (m/z) |
| N-glutamyl | 10.05 | 542.0 | 413.3 (100%) & |
| monascorubraminic | | | 498.3 (50%) |
| acid | | | |
| cis-N-glutaryl | 11.01 | 542.0 | 498.5 (100%) & |
| monascorubraminic | | | 416.0 (35%) |
| acid | | | |
| trans-N-glutaryl | 12.01 | 542.0 | 498.2 (100%) & |
| monascorubraminic | | | 416.0 (30%) |
| acid | | | |
| N-glutaryl | 14.00 | 512.0 | 468.0 (100%) & |
| monascorubramine | | | 386.0 (30%) |

The MS spectrum from 500-550 m/z and selected reaction monitoring transitions for N-glutaryl monascorubramine are shown in FIG. 12C-D. The MS spectrum from 500-550 m/z and selected reaction monitoring transitions for cis-N-glutaryl monascorubraminic acid are shown in FIG. 13C-D. The MS spectrum for trans-N-glutaryl monascorubraminic acid is shown in FIG. 14C. The MS spectrum for N-glutamyl monascorubraminic acid is shown in FIG. 15C.

### Nuclear Magnetic Resonance (NMR)

Nuclear Magnetic Resonance (NMR) spectra were recorded on a Bruker Avance II spectrometer at 300 MHz (¹H, using tetramethylsilane signal as an internal reference, unless noted otherwise) and at 75 MHz (¹³C). Measurements were performed with the sample dissolved in D₂O.

The cis-N-glutaryl monascorubraminic acid as isolated from the colorant composition as produced by methods described in Example 16 and extracted with ethanol. Analysis of the cis-N-glutaryl monascorubraminic acid by ¹H NMR (D₂O, 300 MHz) is performed using the signal at 4.79 ppm from residual solvent in deuterated D₂O for calibration: ¹H NMR: (D₂O, 300 MHz): 8.50 (s, 1H), 7.14 (s, 1H), 6.68 (s, 1H), 6.57 (d, J=12.0 Hz, 1H), 6.51 (d, J=11.9 Hz, 1H), 4.93 (signal overlapping solvent signal, 1H), 2.92-2.72 (m, 2H), 2.67-2.47 (m, 1H), 2.44-2.18 (m, 3H), 1.74 (s, 3H), 1.67-1.56 (m, 2H), 1.40-1.21 (m, 8H), 0.85 (t, J=6.7 Hz, 3H) ppm.

The spectrum (FIG. 13E) shows the presence of the cis-N-glutaryl monascorubraminic acid, water, and an unknown impurity responsible for a signal at 1.93 ppm (singlet). All samples were prepared by dissolving 9 mg of solid in 0.6 mL of D₂O. The J-couplings of about 12 Hz are indicative of the cis-configuration of the double bond.

Both the 2D ¹H¹H-COSY spectrum (FIG. 13F) and the 2D HSQC spectrum (FIG. 13G) of cis-N-glutaryl monascorubraminic acid reveal the methine hydrogen of the glutamic fragment (alpha hydrogen, 2′-H), the signal of which overlaps with that of water at approximately 4.93 ppm in the ¹H NMR spectrum. According to the HSQC spectrum, this hydrogen atom is bonded to a methine carbon with a carbon-13 chemical shift of approximately 69 ppm, consistent with the structure of cis-N-glutaryl monascorubraminic acid.

The trans-N-glutaryl monascorubraminic acid as synthesized by methods described in Example 11 is examined by NMR. FIG. 14E shows the overlapping of the cis-N-glutaryl monascorubraminic acid and trans-N-glutaryl monascorubraminic acid spectra. It can be seen that the core signals of both isomers are the same (0-5 ppm) and the only difference lies in the signals of protons 2 and 3 (as labelled in FIG. 14D), which are the only ones with a different chemical environment (approximately at 6.50 ppm). FIG. 14F shows the focused ¹H NMR of the trans-N-glutaryl monascorubraminic acid. The NMR of the trans isomer indicates J-couplings of 14 Hz indicative of the trans-configuration of the double bond.

### Example 13: Determination of Organic Acids and Sugars in Colorant Compositions

The following protocol describes the methodology used for identification and content of organic acids and sugars: malate, succinate, lactate, acetate, glycerol, ethanol, glucose, fructose and sucrose in colorant compositions herein.

The protocol herein uses a Thermo Scientific Ultimate 3000 RSLC, IPLC system with an autosampler organizer, column manager and heater, quaternary pump with a VWD-3400RS and a RefractoMax 520 (ERC) detector. Data acquisition and system control was performed with Enterprise Chromeleon 7.3 software. The column used was Column Aminex HPX-87H, 300 mm×7.8 mm, 9 um. An analytical balance capable of reading to at least 0.1 mg was used. A membrane filter with 0.22 μm pore size was used.

Instrumental conditions used herein, unless noted otherwise are: Flow: 0.6 mL/min; Detection: IR and UV (210 nm) *To take into account that the flow first passes through the UV detector at 210 nm and then through the IR (Δ~0.2 min); Column temperature: 45 °C; Autosampler temperature: 25 °C; Injection volume: 20 μL; Run time: 33 minutes; Mode: Isocratic.

The mobile phase used was 5 mM H₂SO₄ (pH of about 2). The solvent solutions consisted of H₂O:MeOH (50:50) (solvent solution 1) and H₂O milli Q (solvent solution 2).

A calibration curve was prepared by injection 20 μL, in duplicate, of each dilution of the standard mixture to obtain the calibration curve for each of the compounds.

The samples were prepared by adding about 10.0 mg of sample to a 10 mL volumetric flask and dissolving the sample in solvent solution 2.

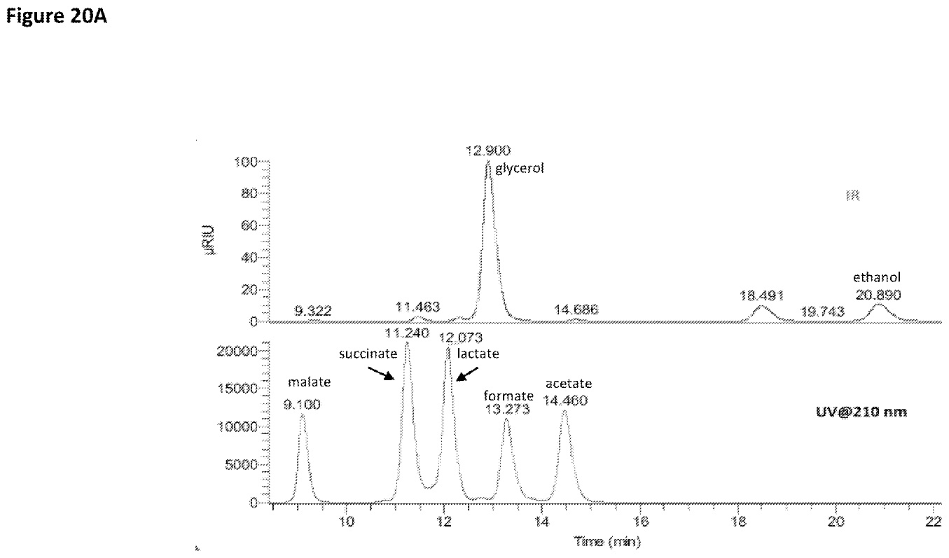

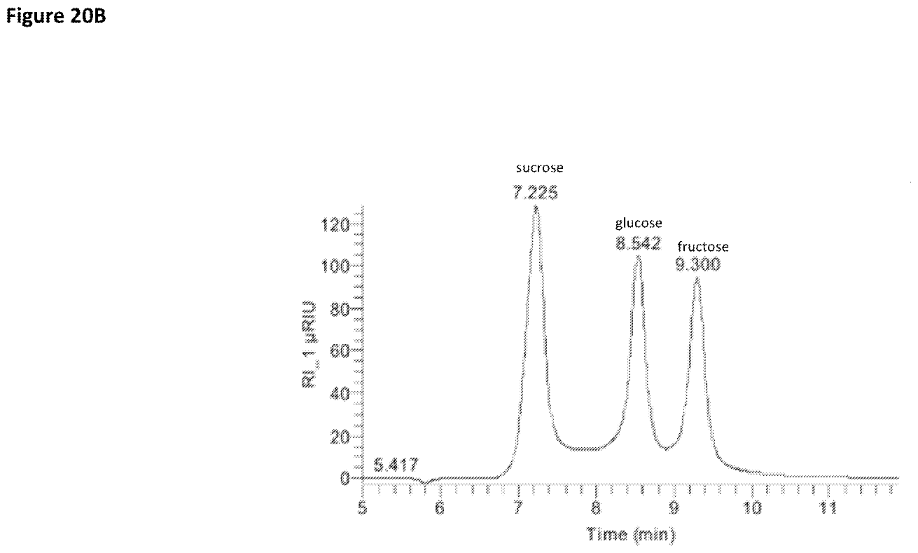

The chromatograms obtained using this method are found in FIG. 20A-B showing determination of malate, succinate, lactate, formate, acetate, glycerol, ethanol, and sucrose, glucose, and fructose respectively.

### Example 14: Comparison of Colorant Compositions

Colorant compositions were made by methods differing from those described herein. In one instance, colorant compositions were prepared using methods described in Rasmussen, K. B. (2015). Talaromyces atroroseus: Genome sequencing, Monascus pigments and azaphilone gene cluster evolution. The colorant properties (e.g., CIELAB) were determined for the colorants prepared by these methods and compared to colorants prepared by the methods described herein, such as in Table 17 showing that the differing methods yield differing colorant properties due to for example, different culturing and/or extraction conditions.

TABLE 17

| | | Red colorant |
|---|---|---|
| | Rasmussen | described herein |
| L* | 70.79 | 78.61 |
| a* | 43.98 | 21.84 |
| b* | 23.60 | 25.50 |
| chroma | 49.9 | 33.57 |
| hue | 28.22 | 49.42 |

### Example 15: Effect of pH on Production of Colorant

Three systems were prepared in a bioreactor containing 2 L of AYP culture medium. The fermentation conditions were 30 °C, 800 RPM, dissolved oxygen over 30%, and pH according to Table 18.

TABLE 18

| System | pH |
|---|---|
| 1 | fixed at pH 5.0 |
| 2 | starting at pH 5.0 without control. The culture |
| | decreases pH down to around 3.7, and then pH is |
| | increased to 5.0 using NaOH 4N when starch depletes. |

The fermentation process takes 72 hours. Absorbance was measured through all the fermentation and used to calculate the titer of colorant (grams of colorant produced per liter of broth) using the next equation (0,0346*absorbance)−0,0000087. The mycelium suspension used in this experiment as inoculum was produced by inoculating 200 mL of MPPY medium in an Erlenmeyer flask with 4×10⁶ conidia and incubating at 28 C and 200 rpm in an orbital shaker for 48 hs.

TABLE 19

| System | Titer (g/L) |
|---|---|
| 1 | 2.41 ± 0.204 |
| 2 | 4.71 ± 0.73 |

As described in Table 19, system 2 leads to the higher titer amount, such as when pH is allowed to decrease throughout the culture before being increased 5. There is no difference in the final biomass dry weight.

### Example 16—Production of Colorant Compositions

### Inoculum Preparation

A sample of a Talaromyces atroroseus conidia suspension was added to 200 mL of sterilized MIPPY (Table 20) in a 1 L flask to a final concentration of 2×10⁵ conidia/mL. The solution was incubated for 24 hours at 30 °C and 130 rpm in an orbital shaker until fungal biomass reaches 2 g/L.

TABLE 20

| Compound | g/L |
|---|---|
| Anhydride glucose | 40 |
| NaNO₃ | 3 |
| Yeast extract | 2 |
| MgSO₄ 7H₂O | 0.5 |
| KCl | 0.5 |
| FeSO₄ 7H₂O | 0.01 |

### Fermentation

The 200 mL inoculum was transferred to the bioreactor containing 1.8 L of DOP culture media (Table 21, 10% v/v). The initial fermentation conditions are 30 °C, pH 5.0, 600 rpm, dissolved oxygen 100%, and aeration 1 vvm. The fermentation step is divided into two stages. The first stage consists of the beginning of the fermentation to starch depletion (approximately 45 hours). During this stage, there is no control of the pH so it decreases from 5.0 to approximately 3.4 and dissolved oxygen decreases to 40%. In stage 2, from 45 hours to the end of the fermentation process (approximately 72 hours), pH is increased back to 5.0 using 2 N NaOH, aeration is lowered to 0.1 vvm, and stirring is set to 800 rpm. Samples are taken two times per day, and the fungal biomass and red colorant composition concentration are measured as dry weight and absorbance, respectively. The dry weight of fungal biomass was measured by filtering the whole broth with cellulose paper with a pore size of 30-40 μm and drying the filter plus the solids at 120 °C for 5 minutes. The absorbance of the fermentation broth was measured by centrifuging the broth at 3000 RPM (1000 g) for 5 minutes and measuring the absorbance at 500 nm of the supernatant. Then, the titer is calculated using the equation (0.0346*absorbance)−0.0000087 which relates absorbance with g/L of red colorant composition. The final titer of the fermentation process is 3.6 g of colorant composition per liter of fermentation broth calculated using the absorbance at 500 nm of the centrifuged fermentation broth.

TABLE 21

| Compounds | g/L |
|---|---|
| Soluble starch | 30 |
| Glucose | 10 |
| Monosodium glutamate | 8.35 |
| Yeast extract | 0.5 |
| MgSO₄•7H₂O | 5 |
| KCl | 0.5 |
| FeSO₄•7H₂O | 0.01 |
| Trace elements (1000 X) | 1 mL/L |

### Ethanol Colorant Composition Extraction

The fungal biomass is removed at 9400 rpm in a continuous stack centrifuge. 300 g of ethanol is added at 96 °C to 125 g of supernatant broth containing the red colorant composition in a 500 mL bottle. The mixture is incubated at 4 °C for at least 24 hours.

The mixture is filtered to separate the precipitate which may be in part composed of exopolysaccharides (EPS) from the supernatant containing the red colorant composition. The supernatant is concentrated by evaporation using a rotary evaporator in a 1 L glass ball at 55 °C, 200 rpm, −0.09 MPa or −27.5 inHg vacuum, for 30 minutes. Sulfuric acid (98%) was added to the concentrated supernatant to lower the pH to 2.5 and boost the precipitation of the red colorant composition, this solution was incubated overnight at 4 °C. The supernatant was separated by centrifugation at 4000 rpm for 10 minutes and the absorbance was measured to calculate the acid precipitation efficiency.

The precipitated red colorant composition was re-suspended in 15 mL of potassium hydroxide (0.1 M) and the volume and absorbance was measured. The red colorant composition was then dried in the oven at 70 °C overnight or sprayed at these conditions with a nozzle air rate of 1.6 bar, air flow of 70 m³/h, inlet temperature of 185 °C, outlet temperature of 74 °C, and feeding rate of 1200 g/h.

The composition of the colorant composition was determined by the methods as described in Examples 12 and 13 to be comprised of ash, succinate, N-glutamyl monascorubraminic acid, cis-N-glutaryl monascorubraminic acid, trans-N-glutaryl monascorubraminic acid, N-glutaryl monascorubramine, and other compounds (see Table 22)

TABLE 22

| Content | Average (%) | Range (%) |
|---|---|---|
| Ash | 3 | — |

| | | | |
|---|---|---|---|
| Succinate | 6.5 | 2 | 12 |
| N-glutamyl | 3.2 | 2 | 6 |
| monascorubraminic | | | |
| acid | | | |
| cis-N-glutaryl | 65.4 | 60 | 75 |
| monascorubraminic | | | |
| acid | | | |
| trans-N-glutaryl | 3.1 | 1 | 7 |
| monascorubraminic | | | |
| acid | | | |
| N-glutaryl | 1.8 | 1 | 4 |
| monascorubramine | | | |
| Other compounds | 17.0 | 7 | 23 |

### Ethyl Acetate Colorant Composition Extraction

The fungal biomass was removed at 9,400 rpm in a continuous stack centrifuge. The supernatant was incubated at 70 °C for 30 minutes to denature and precipitate proteins and exopolysaccharides. The supernatant was filtered through a 40 μm paper filter and the pH was lowered to 2.5 with sulfuric acid (98%). 25 mL of ethyl acetate was added for every 125 g of filtered supernatant and mixed thoroughly at room temperature, resulting in phase separation. The upper ethyl acetate phase containing the red colorant composition was separated from the aqueous phase suing a separatory funnel, the absorbance of the aqueous phase was measured. The red colorant composition was extracted from the 25 mL ethyl acetate phase with 15 mL of a 0.1 M potassium hydroxide solution. The lower water phase from the liquid extraction was recovered and the exact volume and absorbance was measured. The ethyl acetate phase was dried in the oven at 70 °C overnight or spray dried to isolate the dried colorant composition.

### Example 17—Determination of CIELAB Parameters

A solution of the red colorant composition was prepared by diluting the colorant composition in a 50 mM dipotassium phosphate buffer having a pH of about 5.0 in order to obtain an absorbance of about 0.5. The CIELAB parameters of the solution were measured using a CIELAB colorimeter that automatically calculates L*, a*, and b*. The chroma value (C) was calculated from a* and b* using the equation [(a*)²+(b*)²]^{1/2}. The hue value (ω) was calculated from a* and b* using the equation tan⁻¹(a*/b*). The CIELAB parameters using this method were measured for the ethanol extracted colorant composition and pure N-glutaryl monascorubraminic acid. The results of these measurements are found in Table 23.

TABLE 23

| | N-glutaryl | Ethanol |
|---|---|---|
| | monascorubraminic | Extracted |
| | acid | Colorant |
| L* | 78.11 | 78.61 |
| a* | 36.32 | 21.84 |
| b* | 16.12 | 25.50 |
| hue | 23.93 | 33.57 |
| chroma | 39.70 | 49.42 |
| E1% | 71.3 | 250 |

1.-97. (canceled)

98. A method for culturing a pigment-producing fungus, the method comprising the steps of:
(a) inoculating a first liquid media with conidia of the fungus;
(b) performing a mycelium production culturing step comprising culturing the conidia in the first liquid media to generate an inoculum culture comprising mycelium;
(c) inoculating a second liquid media with the inoculum culture or a portion thereof, and
(d) performing a pigment production culturing step comprising culturing the mycelium in the second liquid media to generate a mycelium culture;
wherein the pigment-producing fungus is from the genus Talaromyces, Penicillium, or Monascus, and wherein the second liquid media comprises from 0 to 40 mg/L of phosphate during the second culturing step.

99. The method of claim 98, wherein the fungus is selected from the group consisting of Talaromyces atroroseus, Talaromyces verruculosus, Talaromyces albobiverticillius, Talaromyces purpureogenus, Talaromyces amestolkiae, Talaromyces ruber, Talaromyces flavus, Penicillium purpurogenum, Penicillium oxalicum, Penicillium armenica, Penicillium marneffei, Penicillium atrovenetum, Penicillium rubrum, Monascus purpureus, Monascus ruber, and Monascus pilosus.

100. The method of claim 98, wherein the first liquid media, the second liquid media, or both comprise a mixed nitrogen source.

101. The method of claim 100, wherein the first liquid media comprises a first mixed nitrogen source selected from the group consisting of peptone, tryptone, soy tryptone, yeast extract, one or more proteins, one or more peptides, and two or more amino acids.

102. The method of claim 100, wherein the second liquid media comprises a second mixed nitrogen source selected from the group consisting of peptone, tryptone, soy tryptone, yeast extract, one or more proteins, one or more peptides, and two or more amino acids.

103. The method of claim 98, wherein pH is decreased to about 4.5, about 4.0, about 3.5, about 3.0, or about 2.5 during the pigment production culturing step.

104. The method of claim 98, wherein the dissolved oxygen is maintained at about 30% to 35% in the mycelium production culturing step.

105. The method of claim 98, wherein the dissolved oxygen is maintained at about 30% to 35% in the pigment production culturing step.

106. The method of claim 98, wherein a second nitrogen source is provided during the pigment production culturing step, and the second nitrogen source is one or more amino acids.

107. The method of claim 98, wherein the second liquid media comprises starch.

108. The method of claim 107, wherein the starch is depleted to below 0.1 g/L during the pigment production culturing step, the second liquid media is stirred at a first stirring speed during the pigment production culturing step, and the method further comprises stirring the second liquid media at a second stirring speed after the starch is depleted to below 0.1 g/L during the pigment production culturing step, wherein the second stirring speed is 100 to 300 rpm higher than the first stirring speed.

109. The method of claim 98, wherein at the beginning of the pigment production culturing step the second liquid media comprises a sugar at 8 to 12 g/L, a starch at 25 to 35 g/L, and yeast extract at 0.8 to 1.2 g/L.

110. The method of claim 98, wherein the method further comprises the step of extracting one or more pigments from the mycelium culture to obtain a colorant extract, wherein the one or more pigments are selected from the group consisting of N-glutaryl monascorubraminic acid, N-asparagyl monascorubraminic acid, N-aspartyl monascorubraminic acid, N-cysteinyl monascorubraminic acid, N-phenylalanyl monascorubraminic acid, N-lysyl monascorubraminic acid, N-methionyl monascorubraminic acid, N-glutamyl monascorubraminic acid, and N-arginyl monascorubraminic acid.

111. A colorant composition comprising:
(a) about 60% to about 75% cis-N-glutaryl monascorubraminic acid; and
(b) about 1% to about 7% trans-N-glutaryl monascorubraminic acid.

112. The colorant composition of claim 111, wherein the cis-N-glutaryl monascorubraminic acid is present in the colorant composition in an amount of about 65% and the trans-N-glutaryl monascorubraminic acid is present in the colorant composition in an amount of about 3%.

113. The colorant composition of claim 111, wherein the colorant composition further comprises N-glutamyl monascorubraminic acid in an amount of about 2% to about 6%, preferably in an amount of about 3%.

114. The colorant composition of claim 111, wherein the colorant composition further comprises N-glutaryl monascorubramine in an amount of about 1% to about 4%, preferably in an amount of about 2%.

115. The colorant composition of claim 111, wherein the colorant composition further comprises succinate in an amount of about 2% to about 12%, preferably in an amount of about 7%.

116. A colorant composition comprising:
(a) about 65% cis-N-glutaryl monascorubraminic acid;
(b) about 3% trans-N-glutaryl monascorubraminic acid;
(c) about 3% N-glutaryl monascorubramine;
(d) about 3% N-glutamyl monascorubraminic acid; and
(e) about 7% succinate.
